(19) United States
(12) Patent Application Publication
(10) Pub. No.: US 2019/0211292 A1
(43) Pub. Date: Jul. 11, 2019

(54) PERFUSION BIOREACTOR BAG ASSEMBLIES

(71) Applicant: Juno Therapeutics, Inc., Seattle, WA (US)

(72) Inventors: Pascal BEAUCHESNE, Seattle, WA (US); Chris Duncan VALBURG, Seattle, WA (US)

(73) Assignee: Juno Therapeutics, Inc., Seattle, WA (US)

(21) Appl. No.: 16/331,125

(22) PCT Filed: Sep. 12, 2017
(86) PCT No.: PCT/US2017/051228

§ 371 Date: Mar. 6, 2019

Related U.S. Application Data
(60) Provisional application No. 62/393,583, filed on Sep. 12, 2016.

(51) Int. Cl. C12M 1/00

(52) U.S. Cl. CPC C12M 23/14; C12M 29/10; C12M 29/24

(57) ABSTRACT

The present disclosure is directed to bioreactor bag assemblies that can minimize the amount of additional connections/adaptations made to the bioreactor bag before the bioreactor bag can be used for cell cultivation, thereby reducing the risk of contamination. The bioreactor bag assemblies disclosed herein can include a pre-assembled waste bag connection and pre-assembled tubing arrangements so that the cell media and/or the cell source can be immediately welded to the pre-assembled tubing arrangements.

### CROSS-REFERENCE TO RELATED APPLICATIONS

This application claims priority to U.S. Application No. 62/393,583, filed Sep. 12, 2016, the contents of which are hereby incorporated by reference in their entirety.

### FIELD OF THE DISCLOSURE

This disclosure relates in some aspects to bioreactor bag assemblies, including perfusion bioreactor bag assemblies. More particularly, this disclosure relates in certain aspects to ready-to-use bioreactor bag assemblies that minimize the amount of connections needed to be made by an operator before use and risk of contamination of the bioreactor bag.

### BACKGROUND

Traditional stainless steel systems and piping in manufacturing processes for cell culture have been replaced in many applications by disposable bioreactor bags which in some cases are rocked by a bioreactor rocker. However, there are various types of bioreactor rockers available and each bioreactor rocker system can optionally contain a variety of components with multiple connections. These components can include, for example, a rocking platform optionally with a lid, one or more various containers including cell containers, such as a cellbag, input containers, a pump module, a gas module, and a waste container/bag. In addition, such components can include multiple fluid lines connecting one or more such components to each other and/or to fluid supply connections as well as power cords and data cables. Because the various bioreactor rockers differ from each other, the bioreactor rockers often utilize different and unique bioreactor bags specific for each rocker system.

### SUMMARY

Provided are bioreactor bag assemblies, such as perfusion bioreactor bag assemblies, that can be used across a variety of bioreactor devices. In some aspects, provided are ready-to-use bioreactor bag assemblies with a pre-assembled waste bag connection and pre-assembled tubing arrangements so that the cell media and/or the cell source can be immediately welded to the pre-assembled tubing arrangements. In some embodiments, the bioreactor bag assemblies can minimize the amount of additional connections/adaptations made to the bioreactor bag before the bioreactor bag can be used for cell cultivation. In some embodiments, the bioreactor bag assemblies can minimize the amount of components needed for operation, thereby minimizing the risk of integrity of the bioreactor bag assembly. As such, the bioreactor bag assemblies in some aspects can reduce the risk of contamination and/or loss of product. In addition, minimizing the connections, adaptations, and/or components can allow the bioreactor bag to be compatible with a variety of bioreactor rockers.

Some embodiments include a bioreactor bag assembly that includes a bioreactor bag and a waste bag. In any embodiment, the bioreactor bag can include a bottom surface and a top surface with a plurality of ports, wherein the plurality of ports can include a feed port, a sampling port, and a perfusion port. In any embodiment, the bioreactor bag can include a perfusion filter fluidly connected to the perfusion port. In any embodiment, the waste bag can be fluidly connected to the perfusion port of the bioreactor bag. In any embodiment, the top surface of the bioreactor bag has a first end and a second end opposite the first end, and the perfusion port is closer to the second end than the first end. In any embodiment, the feed port and the sampling port are closer to the first end than the second end. In any embodiment, the top surface of the bioreactor bag has a first side and a second side opposite the first side, and the feed port is closer to the first side than the second side. In any embodiment, the sampling port is closer to the second side than the first side. In any embodiment, the perfusion port is closer to the second side than the first side. In any embodiment, the perfusion filter is inside the bioreactor bag. In any embodiment, the bioreactor bag assembly can include a feed tubing arrangement fluidly connected to the feed port. In any embodiment, the feed tubing arrangement includes polyvinyl chloride (PVC) tubing. In any embodiment, the feed tubing arrangement includes a Y-connector such that the feed tubing arrangement has two inlets. In any embodiment, the bioreactor bag assembly can include a sampling tubing arrangement fluidly connected to the sampling port. In any embodiment, the sampling tubing arrangement can include PVC tubing. In any embodiment, the waste bag can be fluidly connected to the perfusion port via a waste tubing arrangement. In any embodiment, the waste tubing arrangement includes PVC tubing. In any embodiment, the plurality of ports can include a gas inlet port and a gas outlet port. In any embodiment, the top surface of the bioreactor bag has a middle that is halfway between the first end and the second end, and the gas inlet port and the gas outlet port are closer to the middle than the first end or second end. In any embodiment, the plurality of ports includes only the feed port, the sampling port, the perfusion port, the gas inlet port, and the gas outlet port. In any embodiment, the bioreactor bag assembly can include a gas inlet tubing arrangement that includes an inlet filter fluidly connected to the gas inlet port and a gas outlet tubing arrangement that includes an exhaust filter fluidly connected to the gas outlet port.

Some embodiments include a bioreactor system that includes a bioreactor rocker, a bioreactor bag supported on the bioreactor rocker, and a waste bag fluidly connected to the bioreactor bag. In any embodiment, the bioreactor bag includes a bottom surface and a top surface that includes a plurality of ports, wherein the plurality of ports include a feed port, a sampling port, and a perfusion port. In any embodiment, the perfusion filter can be fluidly connected to the perfusion port. In any embodiment, the waste bag can be fluidly connected to the perfusion port of the bioreactor bag. In any embodiment, the bioreactor system can include a feed tubing arrangement fluidly connected to the feed port. In any embodiment, the feed tubing arrangement can include a Y-connector such that the feed tubing arrangement has a first and a second inlet. In any embodiment, a cell media source is fluidly connected to each inlet of the feed tubing arrangement. In any embodiment, each inlet can include PVC and the cell media source can be welded to the PVC of the inlet. In any embodiment, a cell source is fluidly connected to the first inlet and a cell media source is fluidly connected to the second inlet. In any embodiment, each inlet can include PVC and the cell source can be welded to the PVC of the first inlet and the cell media source can be welded to the PVC of the second inlet. In any embodiment, the perfusion filter can be inside the bioreactor bag. In any embodiment, the top surface of the bioreactor bag has a first end and a second end opposite the first end, and the perfusion port is closer to the second end than the first end. In any embodiment, the feed port and the sampling port can be closer to the second end than the first end. In any embodiment, the feed port and the sampling port can be closer to the first end than the second end. In any embodiment, the top surface of the bioreactor bag has a first side and a second side opposite the first side, and the feed port is closer to the first side than the second side. In any embodiment, the sampling port is closer to the second side than the first side and the perfusion port is closer to the second side than the first side. In any embodiment, the bioreactor system can include a sampling tubing arrangement fluidly connected to the sampling port. In any embodiment, the sampling tubing arrangement includes PVC tubing. In any embodiment, the waste bag is fluidly connected to the perfusion port via a waste tubing arrangement. In any embodiment, the waste tubing arrangement includes PVC tubing. In any embodiment, the plurality of ports can include a gas inlet port and a gas outlet port. In any embodiment, the top surface of the bioreactor bag has a middle that is halfway between the first end and the second end, and the gas inlet port and the gas outlet port are closer to the middle than the first end or second end. In any embodiment, the plurality of ports includes only the feed port, the sampling port, the perfusion port, the gas inlet port, and the gas outlet port. In any embodiment, the bioreactor system can include a gas inlet tubing arrangement that includes an inlet filter fluidly connected to the gas inlet port and a gas outlet tubing arrangement that includes an exhaust filter fluidly connected to the gas outlet port.

Some embodiments include a method of using a bioreactor system that includes placing a bioreactor bag of a bioreactor bag assembly on a bioreactor rocker, supplying cell media to the bioreactor bag through a feed port of the bioreactor bag, supplying cells to the bioreactor bag through the feed port, cultivating the cells in the bioreactor bag using agitation provided from the bioreactor rocker, transferring waste filtrate through a perfusion port of the bioreactor bag to a waste bag, and harvesting the cultivated cells. In any embodiment, the bioreactor bag assembly includes a bioreactor bag with a top surface that includes a plurality of ports, wherein the plurality of ports includes a feed port, a sampling port, and a perfusion port. In any embodiment, the bioreactor bag assembly includes a perfusion filter fluidly connected to the perfusion port. In any embodiment, the bioreactor bag assembly includes a waste bag fluidly connected to the perfusion port of the bioreactor bag. In any embodiment, the bioreactor bag assembly includes a feed tubing arrangement fluidly connected to the feed port, wherein the feed tubing arrangement includes a Y-connector such that the feed tubing arrangement has a first inlet and a second inlet. In any embodiment, the cell media is added by welding a cell media source to the first inlet. In any embodiment, the cells are added by welding a cell source to the first inlet. In any embodiment, the cell media is added by welding a cell media source to the first inlet and the cells are added by welding a cell source to the second inlet. In any embodiment, the plurality of ports includes a gas inlet port and a gas outlet port. In any embodiment, the method includes supplying a gas for cell cultivation to the bioreactor bag through the gas inlet port. In any embodiment, the method includes removing a portion of the gas from the bioreactor bag as exhaust through the gas outlet port. In any embodiment, the cultivated cells are harvested by welding a harvest bag to the first or second inlet of the feed tubing and reversing the flow direction of the feed tubing arrangement. In any embodiment, the method includes at least partially inflating the bioreactor bag with a gas through the gas inlet port. In any embodiment, the method includes retrieving a sample of the cultivated cells through the sampling port.

Additional advantages will be readily apparent to those skilled in the art from the following detailed description. The examples and descriptions herein are to be regarded as illustrative in nature and not restrictive.

### BRIEF DESCRIPTION OF THE DRAWINGS

Exemplary embodiments are described with reference to the accompanying figures, in which:

FIG. 1 illustrates an example of a top view of a bioreactor bag disclosed herein.

FIG. 2A illustrates a first example of a feed tubing arrangement for a bioreactor bag assembly disclosed herein.

FIG. 2B illustrates a second example of a feed tubing arrangement for a bioreactor bag assembly disclosed herein.

FIG. 3A illustrates a first example of a sampling tubing arrangement for a bioreactor bag assembly disclosed herein.

FIG. 3B illustrates a second example of a sampling tubing arrangement for a bioreactor bag assembly disclosed herein.

FIG. 4A illustrates a first example of a waste tubing arrangement for a bioreactor bag assembly disclosed herein.

FIG. 4B illustrates a second example of a waste tubing arrangement for a bioreactor bag assembly disclosed herein.

FIG. 5 illustrates an example of a gas outlet tubing arrangement for a bioreactor bag assembly disclosed herein.

FIG. 6 illustrates an example of a gas inlet tubing arrangement for a bioreactor bag assembly disclosed herein.

FIG. 7 illustrates an example of a sampling arrangement for a bioreactor bag assembly disclosed herein.

In the Figures, like reference numbers correspond to like components unless otherwise stated.

### DETAILED DESCRIPTION

### I. Bioreactor Bag

In some embodiments, an exemplary bioreactor bag includes various ports, dip tubes, flaps, and pump tubing among other components. In some embodiments, these components were not delivered in a sealed package, with all necessary components pre-attached. For example, in some embodiments a waste bag and/or feed ports may not be delivered attached to a bioreactor bag and would need to be attached by an operator at the site of use. In some embodiments, additional connections and/or adaptations must be made to the bioreactor bag before the bioreactor bag is used for cell cultivation. For example, the perfusion port may be connected to a waste bag and the user may need to determine a way to connect the cell source and/or media source to the bioreactor bag before proceeding with an experimental run. In some embodiments, each of these components, connections, and adaptions can be a contamination point for the bioreactor bag and associated components. Accordingly, in order to ensure a sterile cell incubation environment, a user may sterilize the completed assembly before an experimental run or risk potential loss of product due to contamination.

The bioreactor bag assemblies described herein can reduce one or more of the above risks, e.g., by providing a ready-to-use sterilized assembly with pre-assembled waste bag connection and pre-assembled PVC tubing so that the cell media and/or cell source can be immediately welded to the PVC tubing. As described in detail below, the bioreactor bag assemblies can minimize the amount of components needed for operation and therefore minimize the various potential points of failure in the bioreactor system and minimize the risk to the integrity of the bioreactor bag. In addition, minimizing the connections, adaptations, and/or components can allow the bioreactor bag to be compatible with a variety of bioreactor rockers.

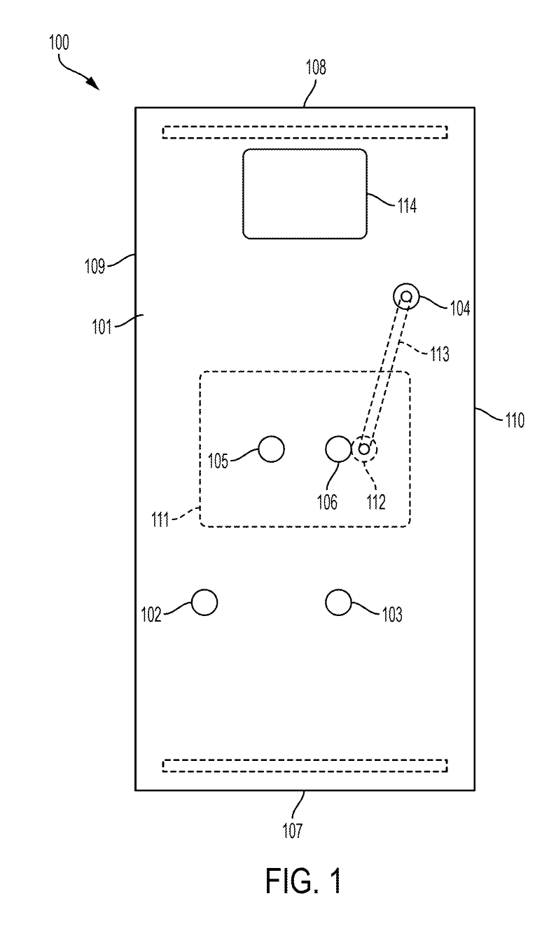

The bioreactor bag assemblies described herein can be used for culturing cells such as the culture of human, animal, insect, and plant cells. In some embodiments, the bioreactor bag assemblies described herein are used in perfusion operations in a bioreactor system. In some embodiments, the bioreactor bag assemblies disclosed herein can be used for clinical cell therapy and T cell applications. FIG. 1 illustrates an example of a top view of bioreactor bag 100. Bioreactor bag 100 can have top surface 101. The top surface can include a plurality of ports. In some embodiments, at least one of the plurality of ports may be placed on a bottom surface of the bioreactor bag, opposite the top surface. The plurality of ports can include a feed port, a sampling port, a perfusion port, a gas inlet port, a DO probe port, a pH probe port, and/or a gas outlet port. In some embodiments, the plurality of ports does not include a DO probe port and/or a pH probe port. In some embodiments, the ports on the top surface only include a feed port, a sampling port, a perfusion port, a gas inlet port, and a gas outlet port. Top surface 101 of bioreactor bag 100 includes feed port 102. A feed port can be used to add various feed components to the bioreactor bag. For example, the feed port can be used to add cells to the bioreactor bag from a cell source and/or add cell media to the bioreactor bag from a cell media source. The cell media can contain nutrients for the cells during cell cultivation. In some embodiments, cell media can be continuously added to the bioreactor bag through the feed port. In some embodiments, the bioreactor bag can be partially filled with cell media and cells through the feed port. In some embodiments, the bioreactor bag may be prefilled with the cell media or prefilled with the cells.

Top surface 101 also includes sampling port 103. A sampling port can be used to remove a sample of the cells and/or other material from the bioreactor bag. For example, during cultivation a user may want to test some of the cells for various characteristics. Accordingly, the user can use the sampling port of the bioreactor bag to obtain the cell sample.

The bioreactor bag can also include a first end and a second end opposite the first end. In addition, the bioreactor bag can include a first side and a second side opposite the first side. For example, top surface 101 of bioreactor bag 100 includes first end 107, second end 108, first side 109, and second side 110. In some embodiments, the sides may be longer than the ends. For example, in some embodiments, the sides may be 558 mm long and the ends may be 275 mm long. In some embodiments, the first end can be the front of the bag when in use and the second end can be the back of the bag when in use. The middle of the top surface of the bag can be halfway between the first end and the second end. The feed port and/or the sampling port can be closer to the first end than the second end. In other embodiments, the feed port and/or the sampling port can be closer to the second end than the first end. In some embodiments, the feed port and/or the sampling port can be closer to the first end than the middle. In some embodiments, the feed port and/or the sampling port can be closer to the second end than the middle. In some embodiments, the feed port and/or the sampling port can be closer to the middle than the first or second end. In some embodiments, the feed port can be closer to the first side than the second side. In some embodiments, the feed port can be closer to the second side than the first side. In addition, the sampling port can be closer to the second side than the first side. In other embodiments, the sampling port can be closer to the first side than the second side.

The bioreactor bag can include perfusion port 104 as shown in FIG. 1. A perfusion port can be used to remove cell waste from the bioreactor bag. For example, as the cells are cultivating in the bioreactor, the cells can produce toxic metabolic by-products. Accordingly, these toxic by-products can be removed through the perfusion port. The bioreactor bag can also include a perfusion filter fluidly connected to the perfusion port. The perfusion filter can allow fluid to be removed from the bioreactor bag with minimal or no cell loss. Accordingly, the filter can have a porosity such that cells cannot pass through it. In some embodiments the perfusion filter can be constructed such that it can move freely on the fluid surface in the bioreactor bag. In some embodiments, the perfusion filter includes a 1.2 micron (pore size) membrane. A filtrate tube can be the connection between the perfusion port and the filtrate port. Accordingly, waste filtrate can exit the bioreactor bag first through the filter to the filtrate tube and then out the perfusion port. In some embodiments, waste filtrate is continuously removed from the bioreactor bag. FIG. 1 includes filter 111 inside bioreactor bag 100, filtrate port 112 on filter 11, and filtrate tube 113 connecting filtrate port 112 and perfusion port 104. The dotted lines of filter 111, filtrate port 112, and filtrate tube 113 signify that these items are not on top surface 101 but inside bioreactor bag 100. The filtrate tube can be a flexible tube that allows the filter to move freely on the fluid surface in the bioreactor bag. In some embodiments, the perfusion port is closer to the second end than the first end of the bioreactor bag as shown in FIG. 1. In other embodiments, the perfusion port is closer to the first end than the second end of the bioreactor bag. In some embodiments, the perfusion port is closer to the first end than the middle. In some embodiments, the perfusion port is closer to the second end than the middle. In some embodiments, the perfusion port is closer to the middle than the first or second end. In addition, the perfusion port can be closer to the second side than the first side. In other embodiments, the perfusion port can be closer to the first side than the second side.

The top surface of the bioreactor bag can also include a gas inlet port and a gas outlet port. For example, FIG. 1 includes gas outlet port 105 and gas inlet port 106 on top surface 111. In some embodiments, the bioreactor bag can be inflated, in some embodiments partially inflated, through the gas inlet port. In some embodiments, gas (e.g., CO₂, oxygen, nitrogen, air, or mixtures thereof) from a gas source can enter through gas inlet port to inflate the bioreactor bag. Besides inflating the bag, the gas can be used for cell cultivation. For example, the gas can be required for cell metabolism. Exhaust gas (e.g., respired gases) from the bioreactor bag can exit the bioreactor bag through the gas outlet port. In some embodiments, the gas inlet port and/or the gas outlet port are closer to the middle than the first end or the second end. In some embodiments, the gas inlet port and/or the gas outlet port are closer to the first end than the middle. In some embodiments, the gas inlet port and/or the gas outlet port are closer to the second end than the middle. In some embodiments, the gas inlet port is closer to the first side than the second side. In some embodiments, the gas inlet port is closer to the second side than the first side. In some embodiments, the gas outlet port is closer to the second side than the first side. In some embodiments, the gas outlet port is closer to the first side than the second side.

At least one of the plurality of ports of the bioreactor bag can have a dip tube(s). In some embodiments, the ports do not include dip tubes. In some embodiments, the bioreactor bag can include flaps (i.e., excess plastic) on at least one of the ends and/or sides of the bioreactor bag. In some embodiments, one or more of these flaps are removed during manufacture of the bioreactor bag and associated components. In other embodiments, the bioreactor bag does not include flaps on the ends and/or sides of the bag. Removal of one or more of the flaps, or a lack of flaps on the bag, can allow the bioreactor bags to fit on various different bioreactor rockers.

The top surface of the bioreactor bag can also include a product label. For example, FIG. 1 includes product label 114 on top surface 101. The bioreactor bag can also include a bottom surface (not shown). In some embodiments, the bottom surface of the bioreactor bag can include at least one of the plurality of ports described above. In other embodiments, the bottom surface can be smooth. In some embodiments, the bioreactor bag is placed on a bioreactor rocker. A bioreactor rocker can rock or agitate the bioreactor bag, thereby providing movement (e.g., aeration and mixing) of the cells in the bag to foster cell cultivation. The rocking or agitation of the bioreactor can also provide efficient gas exchange from the gas-liquid surface. Examples of bioreactors with rocking motion platforms compatible with the bioreactor bag assemblies disclosed herein include, but are not limited to, GE Xuri W25, GE Xuri W5, Sartorius BioSTAT RM 20 | 50, Finesse SmartRocker Bioreactor Systems, and Pall XRS Bioreactor Systems.

The bioreactor bag assemblies disclosed herein can be a disposable single use bioreactor bag assembly. The bioreactor bag can be made out of a flexible material such as a polymeric material (e.g., polyethylene). The polymeric material can be a plastic film or laminate. In addition, the flexible material can include inorganic oxides and/or metals. In some embodiments, the bioreactor bags can be made out of S80 film material. In some embodiments, the bioreactor bags have a gas barrier layer. The gas barrier layer material can include EVOH. In some embodiments, the bioreactor bags can have a product contact layer. The product contact layer material can include polyethylene such as linear low density polyethylene (LLDPE). In some embodiments, the bioreactor bag can be made out of the same material as a Sartorius Flexsafe RM Perfusion Bag. The bioreactor bags can have a total volume from 1-200 L (e.g., 1, 2, 10, 20, 50, 100, or 200 L). The bioreactor bags can have a culture volume of 100 mL to 100 L (e.g., 0.1-0.5 L, 0.2-1 L, 1-5 L, 2-10 L, 5-25 L, 10-50 L, or 20-10 L).

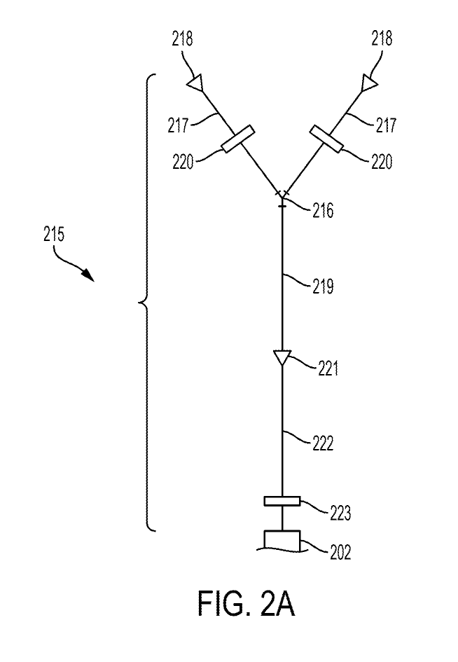

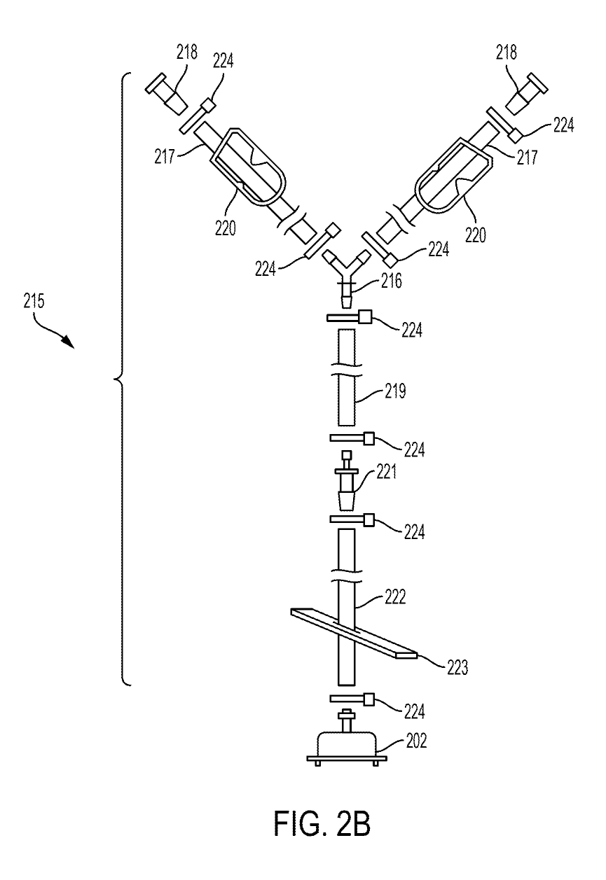

The bioreactor bag assembly can also include a feed tubing arrangement fluidly connected to the feed port. The feed tubing arrangement can provide various feed components (e.g., cells and/or media) to the bioreactor bag's feed port. FIGS. 2A-B illustrate examples of feed tubing arrangement 215 fluidly connected to feed port 202 a bioreactor bag assembly disclosed herein. The feed tubing arrangement can include Y-connector 216. A Y-connector can be used so that the feed tubing arrangement has two inlets. Accordingly, a cell media source can be fluidly connected to one or each inlet of the feed tubing arrangement. In some embodiments, a cell source can be fluidly connected to one or each inlet of the feed tubing arrangement. In other embodiments, a cell source can be connected to one inlet and a media source can be connected to the other inlet. In some embodiments, the Y-connector can be suitable for PVC tubing. The length of the tubing in the feed tubing arrangement can be such that there is sufficient length to reach a feed source (e.g., cell source and/or media source). For example, a feed source may be connected to an IV pole and there should be sufficient tubing length such that the feed tubing arrangement can reach the feed source.

The feed tubing arrangement can also include feed inlet tubing. For example, FIGS. 2A-B include feed inlet tubing 217. The feed inlet tubing can be silicone tubing and/or polyvinyl chloride (PVC) tubing. In some embodiments, the feed inlet tubing is PVC tubing. In some embodiments, the feed inlet tubing is 0.118×0.164 PVC tubing. In some embodiments, the feed inlet tubing is Tygon® 0.118×0.164 tubing. In some embodiments, the feed inlet tubing is such that additional elements can be welded to the feed inlet tubing. For example, a cell source and/or a cell media source can be welded to the feed inlet tubing such that cells and/or cell media can be supplied to the bioreactor bag through the feed port. In some embodiments, the feed inlet tubing can meet Terumo TSCD-II welder specifications. In some embodiments, the feed inlet tubing can be compatible with standard PVC blood collection type tubing. In some embodiments, the feed inlet tubing has an ID: 2.9-3.1 mm/OD: 3.9-4.5 mm. In some embodiments, the feed inlet tubing has an ID: 3 mm/OD: 4.17 mm. One or both sections of the feed inlet tubing can be about 350-550 mm, 400-500 mm, 425-475 mm, or 460 mm in length. The feed tubing arrangement can also include plugs 218. The plugs can be plugs for PVC tubing. In some embodiments, the plugs are press-in plugs 3/32. The feed tubing arrangement can also include an inlet clamp(s) on the feed inlet tubing. FIGS. 2A-B show clamps 220. In some embodiments, the inlet clamps are pinch clamps and/or slide clamps. In some embodiments, the inlet clamps are suitable for PVC tubing.

The feed tubing arrangement can also include post-Y-connector tubing. FIGS. 2A-2B include post-Y-connector tubing 219. The post-Y-connector tubing can be silicone tubing and/or polyvinyl chloride (PVC) tubing. In some embodiments, the post-Y-connector tubing is PVC tubing. In some embodiments, the post-Y-connector tubing is 0.118×0.164 PVC tubing. In some embodiments, the post-Y-connector tubing is Tygon® 0.118×0.164 tubing. In some embodiments, the post-Y-connector tubing is such that additional elements can be welded to the post-Y-connector tubing. For example, a cell source and/or a cell media source can be welded to the post-Y-connector tubing such that cells and/or cell media can be supplied to the bioreactor bag through the feed port. In some embodiments, the post-Y-connector tubing can meet Terumo TSCD-II welder specifications. In some embodiments, the post-Y-connector tubing can be compatible with standard PVC blood collection type tubing. In some embodiments, the post-Y-connector tubing has an ID: 2.9-3.1 mm/OD: 3.9-4.5 mm. In some embodiments, the post-Y-connector tubing has an ID: 3 mm/OD: 4.17 mm. The post-Y-connector tubing can be about 200-400 mm, 250-350 mm, 275-325 mm, or 300 mm in length.

The feed tubing arrangement can also include a reducer as shown in FIGS. 2A-B as reducer 221. The reducer can be a classic series barbs. In some embodiments, the reducer is a 1/8×5/32 reducer. In some embodiments, the reducer is Value Plastics #3040-6005.

The feed tubing arrangement can include post-reducer tubing as shown in FIGS. 2A-2B as post-reducer tubing 222. The post-reducer tubing can be silicone tubing and/or polyvinyl chloride (PVC) tubing. In some embodiments, the post-reducer tubing is silicone tubing. In some embodiments, the post-reducer tubing is ⅛×¼ silicone tubing. The post-reducer tubing can be about 1000-1500 mm, 1250-1450 mm, 1350-1400 mm, or 1380 mm in length. The feed tubing arrangement can include a clamp on the post-reducer tubing. FIGS. 2A-B illustrate post-reducer clamp 223. The post-reducer clamp can be a pinch clamp or a slide clamp. In some embodiments, the post-reducer clamp is suitable for silicone tubing.

A portion of tubing of the feed tubing arrangement can be connected to a pump or multiple pumps such that fluids connected to the feed inlet tubing can be transferred to the bioreactor bag through the feed port. In some embodiments, silicone tubing of the feed tubing arrangement is connected to the pump. In some embodiments, the post-reducer tubing is connected to a pump. In some embodiments, gravity can provide the force for fluids connected to the feed inlet tubing to be transferred to the bioreactor bag through the feed port.

The connectors used in the feed tubing arrangement can be barbed connectors and/or luer lock connectors. In some embodiments, only barbed connectors are used. In some embodiments, at least some of the connections of the feed tubing arrangement can have cable ties, or other ties to secure the connection, on them. In other embodiments, cable ties can be on every connection of the feed tubing arrangement as shown in FIG. 2B with cable ties 224. In addition, all the connections of the feed tubing arrangement can be fluidly connected.

In some embodiments, the feed tubing arrangement can also be used for harvesting cultivated cells. For example, a harvest bag can be welded to the feed inlet tubing and/or the post-Y-connector tubing. In some embodiments, the harvest bag is welded to the PVC tubing of the feed inlet tubing and/or the PVC tubing of the post-Y-connector tubing. To harvest the cells, the flow of the feed tubing arrangement can be reversed. Gravity can provide sufficient force to drive the flow of cells from the bioreactor bag to the harvest bag. In other embodiments, a pump can be used to drive the flow of cells from the bioreactor bag to the harvest bag as explained above.

The bioreactor bag assembly can also include a sampling tubing arrangement fluidly connected to the sampling port. The sampling tubing arrangement can be used to remove a sample of the cells and/or other material from the bioreactor bag. For example, during cultivation, a user may want to test some of the cells for various characteristics. Accordingly, the user can use the sampling tubing arrangement fluidly connected to the sampling port of the bioreactor bag to obtain the cell sample.

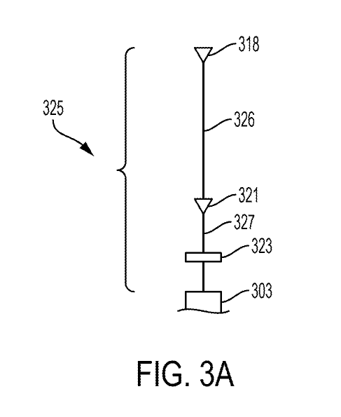

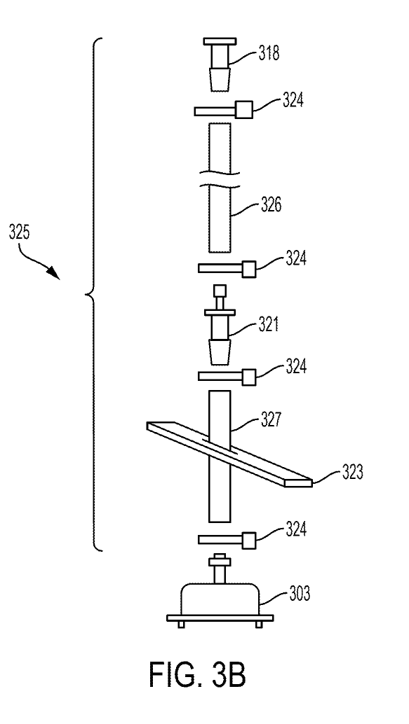

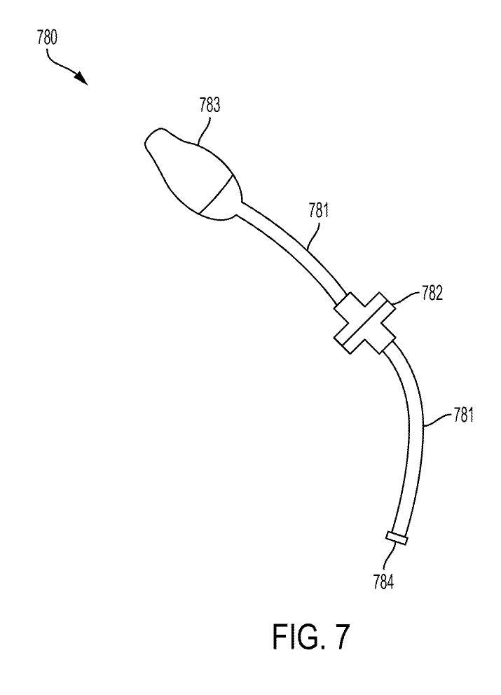

FIGS. 3A-B illustrate examples of sampling tubing arrangement 325 fluidly connected to sampling port 303 for a bioreactor bag assembly disclosed herein. The sampling tubing arrangement can include sampling inlet tubing. For example, FIGS. 3A-B include sampling inlet tubing 326. The sampling inlet tubing can be silicone tubing and/or polyvinyl chloride (PVC) tubing. In some embodiments, the sampling inlet tubing is PVC tubing. In some embodiments, the sampling inlet tubing is 0.118×0.164 PVC tubing. In some embodiments, the sampling inlet tubing is Tygon® 0.118×0.164 tubing. In some embodiments, the sampling inlet tubing is such that additional elements can be welded to the sampling inlet tubing. For example, a sampling arrangement can be welded to the sampling inlet tubing such that sample cells can be removed through the sampling port and sampling tubing arrangement. FIG. 7 illustrates an example of sampling arrangement 780 that can be welded to a bioreactor bag assembly disclosed herein. The sampling arrangement can include tubing (781), a 1-way check valve (782), and microclave reusable sampling port (783). The tubing of the sampling arrangement can be silicone tubing and/or polyvinyl chloride (PVC) tubing. In some embodiments, the tubing is PVC tubing. In some embodiments, the tubing is 0.118×0.164 PVC tubing. In some embodiments, the tubing is Tygon® 0.118×0.164 tubing. In some embodiments, the tubing can meet Terumo TSCD-II welder specifications. In some embodiments, the tubing can be compatible with standard PVC blood collection type tubing. In some embodiments, the tubing has an ID: 2.9-3.1 mm/OD: 3.9-4.5 mm. In some embodiments, the tubing has an ID: 3 mm/OD: 4.17 mm. The end (784) of the tubing opposite the microclave reusable sampling port can be sealed. The sampling arrangement can be welded to the sampling inlet tubing of a bioreactor bag assembly to enable sampling. The sampling arrangement can be about 20-40 mm, 25-35 mm, or 30 mm in length. The sampling arrangement can also have a volume of about 1-5 mL, 2-3 mL, or 2.2 mL. At least some of the connections of the sampling arrangement can be bonded to prevent leaks. In some embodiments, all the connections of the sampling arrangement are bonded to prevent leaks. The microclave reusable sampling port can be bonded to prevent accidental unscrewing of the microclave sampling port. The microclave reusable sampling port can be connected to a sampling syringe in order to remove a sample from the bioreactor bag. The 1-way check valve can prevent accidental backflow during sampling and can protect the culture from contaminations. As such, a user may not be able to push back any material from a sampling syringe into the culture bag. In addition, the line can be flushed between samples by pulling gas from the headspace of the culture bag when the bioreactor bag is placed flat on a bioreactor rocker.

In some embodiments, the sampling inlet tubing can meet Terumo TSCD-II welder specifications. In some embodiments, the sampling inlet tubing can be compatible with standard PVC blood collection type tubing. In some embodiments, the sampling inlet tubing has an ID: 2.9-3.1 mm/OD: 3.9-4.5 mm. In some embodiments, the sampling inlet tubing has an ID: 3 mm/OD: 4.17 mm. The sampling inlet tubing can be about 200-400 mm, 250-350 mm, 275-325 mm, or 300 mm in length. The sampling tubing arrangement can also include plug 318. The plug can be a plug for PVC tubing. In some embodiments, the plug is a press-in plug 3/32.

The sampling tubing arrangement can also include a reducer as shown in FIGS. 3A-B as reducer 321. The reducer can be a classic series barbs. In some embodiments, the reducer is a 1/8×5/32 reducer. In some embodiments, the reducer is Value Plastics #3040-6005.

The sampling tubing arrangement can include post-reducer sampling tubing as shown in FIGS. 3A-B as post-reducer sampling tubing 327. The post-reducer sampling tubing can be silicone tubing and/or polyvinyl chloride (PVC) tubing. In some embodiments, the post-reducer sampling tubing is silicone tubing. In some embodiments, the post-reducer sampling tubing is ⅛×¼ silicone tubing. The post-reducer sampling tubing can be about 10-100 mm, 25-75 mm, 40-60 mm, or 50 mm in length. The sampling tubing arrangement can include a clamp on the post-reducer sampling tubing. FIGS. 3A-B illustrate post-reducer sampling clamp 323. The post-reducer sampling clamp can be a pinch clamp or a slide clamp. In some embodiments, the post-reducer sampling clamp is suitable for silicone tubing.

A portion of tubing of the sampling tubing arrangement can be connected to a pump or multiple pumps such that sample fluids can be removed from the sampling port. In some embodiments, silicone tubing of the sampling tubing arrangement is connected to the pump. In some embodiments, the post-reducer sampling tubing is connected to a pump. In some embodiments, gravity can provide the force for sample fluids to be removed from the sampling port.

The connectors used in the sampling tubing arrangement can be barbed connectors and/or luer lock connectors. In some embodiments, only barbed connectors are used. In some embodiments, at least some of the connections of the sampling tubing arrangement can have cable ties, or other ties to secure the connection, on them. In other embodiments, cable ties can be on every connection of the sampling tubing arrangement as shown in FIG. 3B with cable ties 324. In addition, all the connections of the feed tubing arrangement can be fluidly connected.

The bioreactor bag assembly can also include a waste bag fluidly connected to the perfusion port of the bioreactor bag. The waste bag can store the cell waste removed from the bioreactor bag. Accordingly, waste filtrate can exit the bioreactor bag out the perfusion port and then be stored in the waste bag.

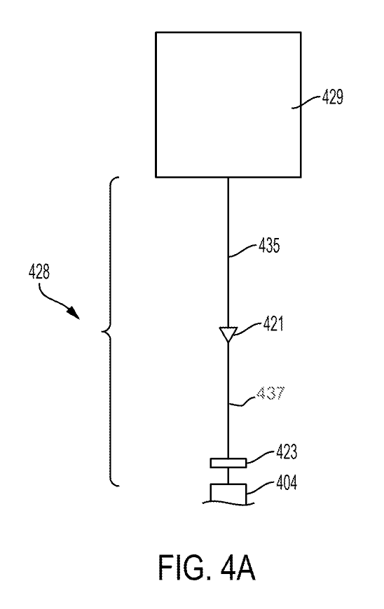

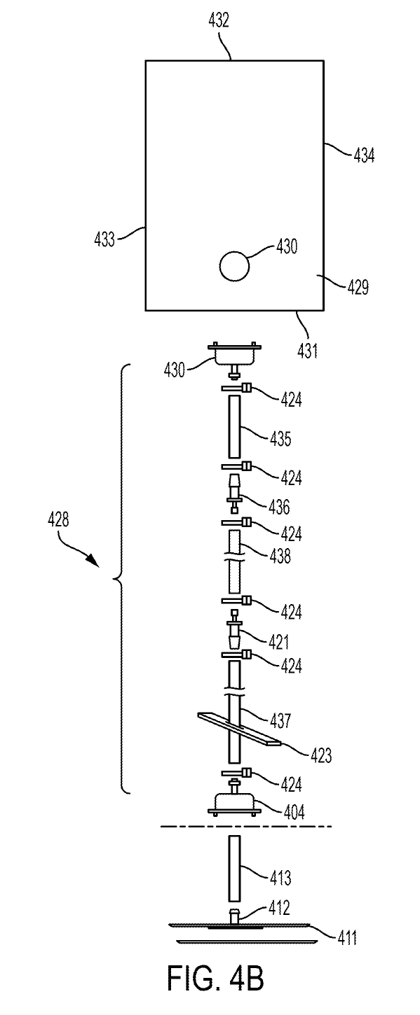

FIGS. 4A-B illustrate examples of waste tubing arrangement 428 fluidly connecting waste bag 429 and perfusion port 404 of the bioreactor bag assembly disclosed herein. As such, waste from the bioreactor bag can travel through filter 411, out filtrate port 412, through filtrate tube 413, out perfusion port 404, and then into waste bag 429. The length of the tubing in the waste tubing arrangement can be such that there is sufficient length for the waste bag to be on the same or different platform of a bioreactor table or console from the bioreactor bag. For example, the waste bag might be on a shelf or platform below the bioreactor rocker (with bioreactor bag) and there should be sufficient tubing length such that the waste tubing arrangement can reach the shelf or platform. The waste bag can have a total volume from 1-200 L (e.g., 1, 2, 10, 20, 50, 100, or 200 L). In some embodiments, the waste bag is a 10 L bag. The waste bag can have waste port. In some embodiments, the waste port is on a top surface of the waste bag as shown in FIG. 4B as waste port 430. Having the waste port on a top surface of the waste bag can allow the waste bag to be on a shelf or platform below the bioreactor bag and the tubing from the bioreactor bag from the waste bag to be vertically aligned without having to bend the tubing. The waste bag can also have a first end and a second end opposite the first end. In addition, the waste bag can have a first side and a second side opposite the first side. For example, waste bag 429 can include first end 431, second end 432, first side 433, and second side 434 as shown in FIG. 4B. In some embodiments, the sides of the waste bag may be longer than the ends. For example, the sides may be 500 mm long and the ends may be 380 mm long. In some embodiments, the first end can be the front of the waste bag when in use and the second end can be the back of the waste bag when in use. In some embodiments, the waste port is on the first end of the waste bag. The middle of the top surface of the waste bag can be halfway between the first end and the second end. The waste port can be closer to the first end than the second end. In other embodiments, the waste port can be closer to the second end than the first end. In some embodiments, the waste port can be closer to the first end than the middle. In some embodiments, the waste port can be closer to the second end than the middle. In some embodiments, the waste port can be closer to the middle than the first or second end. In some embodiments, the waste port can be closer to the first side than the second side. In some embodiments, the waste port can be closer to the second side than the first side.

The waste tubing arrangement can include waste bag inlet tubing. For example, FIGS. 4A-B include waste bag inlet tubing 435. The waste bag inlet tubing can be silicone tubing and/or polyvinyl chloride (PVC) tubing. In some embodiments, the waste bag inlet tubing is PVC tubing. In some embodiments, the waste bag inlet tubing is silicone tubing. In some embodiments, the waste bag inlet tubing is 0.118×0.164 PVC tubing. In some embodiments, the waste bag inlet tubing is Tygon® 0.118×0.164 tubing. In some embodiments, the waste bag inlet tubing is ⅛×¼ silicone tubing. In some embodiments, the waste bag inlet tubing is such that additional elements can be welded to the waste bag inlet tubing such as additional waste bags. In some embodiments, the waste bag inlet tubing can meet Terumo TSCD-II welder specifications. In some embodiments, the waste bag inlet tubing can be compatible with standard PVC blood collection type tubing. In some embodiments, the waste bag inlet tubing has an ID: 2.9-3.1 mm/OD: 3.9-4.5 mm. In some embodiments, the waste bag inlet tubing has an ID: 3 mm/OD: 4.17 mm. The waste bag inlet tubing can be about 10-200 mm, 25-300 mm, 25-75 mm, or 50 mm in length.

The waste tubing arrangement can also include a reducer as shown in FIGS. 4A-4B as reducer 421. The waste tubing arrangement can also include a second reducer as shown in FIG. 4B as reducer 436. The reducer(s) can be a classic series barbs. In some embodiments, the reducer(s) is a 1/8×5/32 reducer. In some embodiments, the reducer(s) is Value Plastics #3040-6005.

The waste tubing arrangement can also include perfusion port outlet tubing. For example, FIGS. 4A-B include perfusion port outlet tubing 437. The perfusion port outlet tubing can be silicone tubing and/or polyvinyl chloride (PVC) tubing. In some embodiments, the perfusion port outlet tubing is silicone tubing. In some embodiments, the perfusion port outlet tubing is ⅛×¼ silicone tubing. The perfusion port outlet tubing can be about 1000-2000 mm, 1250-1750 mm, 1500-1600 mm, or 1520 mm in length. The waste tubing arrangement can include a clamp on the perfusion port outlet tubing. FIGS. 4A-B illustrate perfusion port outlet tubing clamp 423. The perfusion port outlet tubing clamp can be a pinch clamp or a slide clamp. In some embodiments, the post-reducer sampling perfusion port outlet tubing clamp is suitable for silicone tubing.

The waste tubing arrangement can also include intermediate waste tubing. For example, FIG. 4B includes intermediate waste tubing 438. Intermediate waste tubing can be between two other tubing tubes. In some embodiments, intermediate waste tubing is between two reducers. The intermediate waste tubing can be silicone tubing and/or polyvinyl chloride (PVC) tubing. In some embodiments, the intermediate waste tubing is PVC tubing. In some embodiments, the intermediate waste tubing is 0.118×0.164 PVC tubing. In some embodiments, the intermediate waste tubing is Tygon® 0.118×0.164 tubing. In some embodiments, the intermediate waste tubing is such that additional elements can be welded to the intermediate waste tubing such as additional waste bags. In some embodiments, the intermediate waste tubing can meet Terumo TSCD-II welder specifications. In some embodiments, the intermediate waste tubing can be compatible with standard PVC blood collection type tubing. In some embodiments, the intermediate waste tubing has an ID: 2.9-3.1 mm/OD: 3.9-4.5 mm. In some embodiments, the intermediate waste tubing has an ID: 3 mm/OD: 4.17 mm. The intermediate waste tubing can be about 500-1500 mm, 750-1250 mm, 900-1000 mm, or 920 mm in length.

A portion of tubing of the waste tubing arrangement can be connected to a pump or multiple pumps such that waste from the bioreactor bag can be transferred to the waste bag through the perfusion port. In some embodiments, silicone tubing of the waste tubing arrangement is connected to the pump. In some embodiments, the perfusion port outlet tubing and/or the waste bag inlet tubing is connected to a pump. In some embodiments, gravity can provide the force for waste to be removed from the bioreactor bag to be transferred to the waste bag.

The connectors used in the waste tubing arrangement can be barbed connectors and/or luer lock connectors. In some embodiments, only barbed connectors are used. In some embodiments, at least some of the connections of the waste tubing arrangement can have cable ties, or other ties to secure the connection, on them. In other embodiments, cable ties can be on every connection of the waste tubing arrangement as shown in FIG. 4B with cable ties 424. In addition, all the connections of the waste tubing arrangement can be fluidly connected.

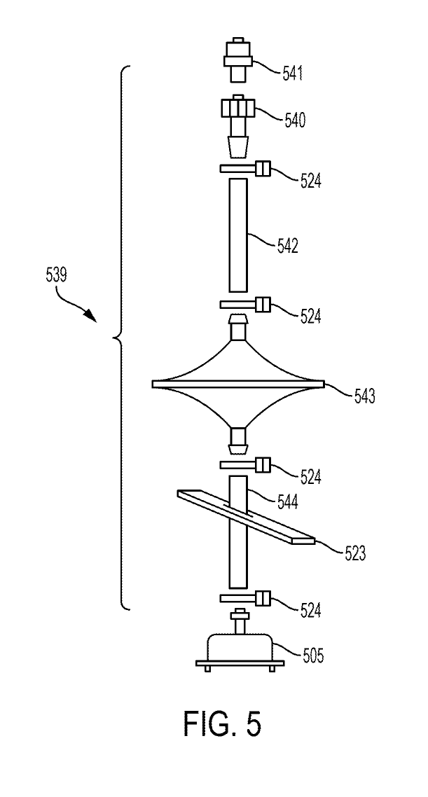

The bioreactor bag assembly can also include a gas outlet tubing arrangement fluidly connected to the gas outlet port of the bioreactor bag. The gas outlet tubing arrangement can allow exhaust gas from the bioreactor bag to be vented. FIG. 5 illustrates an example of gas outlet tubing arrangement 539 fluidly connected to gas outlet port 505 of the bioreactor bag. The gas outlet tubing arrangement can include an exhaust filter. The exhaust filter can ensure that no cells and/or media are released as an aerosol from the bioreactor bag. In addition, the exhaust filter can also ensure that any backflow through the exhaust filter would not result in contamination of the cell culture in the bioreactor bag. FIG. 5 illustrates filter 543. The gas outlet tubing arrangement can also include an exhaust filter inlet tubing and an exhaust filter outlet tubing. The exhaust filter can be between exhaust filter inlet tubing and exhaust filter outlet tubing. For example, FIG. 5 illustrates exhaust filter 543 between exhaust filter inlet tubing 544 and exhaust filter outlet tubing 542.

The exhaust filter inlet tubing can be silicone tubing and/or polyvinyl chloride (PVC) tubing. In some embodiments, the exhaust filter inlet tubing is silicone tubing. In some embodiments, the exhaust filter inlet tubing is 3/16×5/16 silicone tubing. The exhaust filter inlet tubing can be about 10-100 mm, 25-75 mm, 40-60 mm, or 50 mm in length. The gas outlet tubing arrangement can include a clamp on the exhaust filter inlet tubing. FIG. 5 illustrates exhaust filter inlet tubing clamp 523. The exhaust filter inlet tubing clamp can be a pinch clamp or a slide clamp. In some embodiments, the exhaust filter inlet tubing clamp is suitable for silicone tubing.

The exhaust filter outlet tubing can be silicone tubing and/or polyvinyl chloride (PVC) tubing. In some embodiments, the exhaust filter outlet tubing is silicone tubing. In some embodiments, the exhaust filter outlet tubing is 3/16×5/16 silicone tubing. The exhaust filter outlet tubing can be about 10-100 mm, 25-75 mm, 40-60 mm, or 50 mm in length. The gas outlet tubing arrangement can include a clamp on the exhaust filter outlet tubing. The exhaust filter outlet tubing clamp can be a pinch clamp or a slide clamp. In some embodiments, the exhaust filter outlet tubing clamp is suitable for silicone tubing.

The gas outlet tubing arrangement can also include an M-luer 3/16 (541) and a check valve (540) as shown in FIG. 5. The connectors used in the gas outlet tubing arrangement can be barbed connectors and/or luer lock connectors. In some embodiments, only barbed connectors are used. In some embodiments, at least some of the connections of the gas outlet tubing arrangement can have cable ties, or other ties to secure the connection, on them. In other embodiments, cable ties can be on every connection of the gas outlet tubing arrangement as shown in FIG. 5 with cable ties 524. In addition, all the connections of the gas outlet tubing arrangement can be fluidly connected.

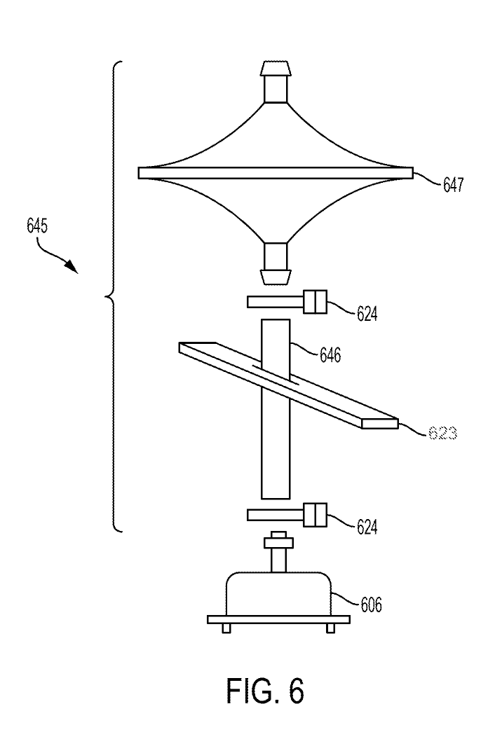

The bioreactor bag assembly can also include a gas inlet tubing arrangement fluidly connected to the gas inlet port of the bioreactor bag. The gas inlet tubing arrangement can be connected to a gas source and allow the gas from a gas source to enter the gas inlet port. FIG. 6 illustrates an example of gas inlet tubing arrangement 645 fluidly connected to gas inlet port 606 of the bioreactor bag. The gas inlet tubing arrangement can include an inlet filter. The inlet filter can be a sterilizing inlet filter. FIG. 6 illustrates inlet filter 647. The gas inlet tubing arrangement can also include gas inlet tubing. For example, FIG. 6 illustrates gas inlet tubing 646.

The gas inlet tubing can be silicone tubing and/or polyvinyl chloride (PVC) tubing. In some embodiments, the gas inlet tubing is silicone tubing. In some embodiments, the gas inlet tubing is 3/16×5/16 silicone tubing. The gas inlet tubing can be about 10-100 mm, 25-75 mm, 40-60 mm, or 50 mm in length. The gas inlet tubing arrangement can include a clamp on the gas inlet tubing. FIG. 6 illustrates gas inlet tubing clamp 623. The gas inlet tubing clamp can be a pinch clamp or a slide clamp. In some embodiments, the gas inlet tubing clamp is suitable for silicone tubing.

The connectors used in the gas inlet tubing arrangement can be barbed connectors and/or luer lock connectors. In some embodiments, only barbed connectors are used. In some embodiments, at least some of the connections of the gas inlet tubing arrangement can have cable ties, or other ties to secure the connection, on them. In other embodiments, cable ties can be on every connection of the gas inlet tubing arrangement as shown in FIG. 6 with cable ties 624. In addition, all the connections of the gas inlet tubing arrangement can be fluidly connected.

The bioreactor bag assemblies disclosed herein can be sterile. For example, the bioreactor bag assemblies may be irradiated with ionizing radiation such as gamma radiation, electron beam, or high energy x-rays using a dose to ensure sterility of the bioreactor bag assembly. In addition, all of the various components of the bioreactor bag assembly (e.g., the bioreactor bag, the ports, the tubing arrangements, the waste bag, the connectors, the filters, etc.) can be constructed from radiation-resistant materials, e.g., ethylene copolymers, silicones, styrene copolymers, polysulfones etc. In some embodiments, the bioreactor bag assemblies disclosed herein can be sterilized and ready-for-use without additional sterilization. In some embodiments, the bioreactor bag assemblies are sealed in a sterilized state (i.e., no open tubing). In some embodiments, the bioreactor bag assemblies can include plugs, seals, or clamps on tubing to prevent open tubing.

In some embodiments, the PVC tubing disclosed herein can be DEHP-free PVC weldable tubing. In some embodiments, the silicone tubing disclosed herein can be silicone pump tubing. Furthermore, the bioreactor bag assemblies can preferably include barbed connections. In some instances, luer lock connections can be insufficiently tightened or accidentally disconnected, thereby compromising the integrity of the bioreactor bag assembly.

### II. Method of Culturing and Processing Cells

In some embodiments, the bioreactor bag assemblies provided herein, such as perfusion bioreactor bag assemblies, can be used for culturing cells, such as in connection with manufacturing, generating or producing a cell therapy. In some embodiments, the cell therapy includes cells, such as T cells, engineered with a recombinant receptor, such as a chimeric antigen receptor, e.g. CAR T cells. In some embodiments, the culturing is carried out under conditions for cultivation and/or expansion of the cells, e.g. stimulation of the cells, for example, to induce their proliferation and/or activation.

In some embodiments, culturing cells using the bioreactor bag assemblies, such as in connection with manufacturing, generating or producing a cell therapy, can be carried out via a process that includes one or more further processing steps, such as steps for the isolation, separation, selection, activation or stimulation, transduction, washing, suspension, dilution, concentration, and/or formulation of the cells. In some embodiments, the methods of generating or producing a cell therapy include isolating cells from a subject, preparing, processing, culturing under one or stimulating conditions, wherein at least a portion of the culturing is carried out using the provided bioreactor bag assemblies, and/or engineering (e.g. transducing) the cells. In some embodiments, the method includes processing steps carried out in an order in which: cells, e.g. primary cells, are first isolated, such as selected or separated, from a biological sample; selected cells are incubated with viral vector particles for transduction, optionally subsequent to a step of stimulating the isolated cells in the presence of a stimulation reagent; culturing the transduced cells, such as to expand the cells; and formulating the transduced cells in a composition. In some embodiments, the generated engineered cells are re-introduced into the same subject, before or after cryopreservation.

In some embodiments, the provided methods are carried out such that one, more, or all steps in the preparation of cells for clinical use, e.g., in adoptive cell therapy, are carried out without exposing the cells to non-sterile conditions and without the need to use a sterile room or cabinet. In some embodiments of such a process, the cells are isolated, separated or selected, transduced, washed, optionally activated or stimulated and formulated, all within a closed system. In some embodiments, the closed system is or includes bioreactor bag assemblies described herein, such as perfusion bioreactor bag assemblies. In some embodiments, the methods are carried out in an automated fashion. In some embodiments, one or more of the steps is carried out apart from the closed system or device.

In some embodiments, the bioreactor bag assemblies described herein provides for a closed system for expansion of the cells that can be integrated into known cell expansion systems and/or into systems for carrying out one or more of the other processing steps of a method for manufacturing, generating or producing a cell therapy. In some embodiments, one or more or all of the processing steps, e.g., isolation, selection and/or enrichment, processing, incubation in connection with transduction and engineering, and formulation steps is carried out using a system, device, or apparatus in an integrated or self-contained system, and/or in an automated or programmable fashion. In some aspects, the system or apparatus includes a computer and/or computer program in communication with the system or apparatus, which allows a user to program, control, assess the outcome of, and/or adjust various aspects of the processing, isolation, engineering, and formulation steps. In one example, the system is a system as described in International Patent Application, Publication Number WO2009/072003, or US 20110003380 A1. In one example, the system is a system as described in International Publication Number WO2016/073602.

A. Culturing Cells

In some embodiments, the bioreactor bag assemblies provided herein, such as perfusion bioreactor bag assemblies, can be filled, e.g. via the feed port, with cell media and/or cells for culturing of the added cells. The cells can be from any cell source for which culture of the cells is desired, for example, for expansion and/or proliferation of the cells. In some embodiments, the cells are or contain immune cells, such as primary cells obtained from a subject or derived from primary cells obtained from a subject. In some aspects, the cells are or contain T cells or NK cells. In some embodiments, the cells comprise CD4+ and CD8+ T cells. In some embodiments, the cells comprise CD4+ or CD8+ T cells. In some embodiments, the cells for culture are cells that are or have been engineered, e.g. transduced, to express a recombinant receptor, such as a chimeric antigen receptor (CAR), such as generated in accord with one or more processing step for manufacturing, producing or generating a cell therapy. Exemplary of such transduced cells, and the one or more processing steps, are described below.

In some embodiments, the bioreactor bag assembly is configured for integration and or operable connection and/or is integrated or operably connected, to a closed system or device that carries out one or more processing steps. In one example, the system is a system as described in International Publication Number WO2016/073602. In some embodiments, the system includes a centrifugal chamber and the process includes effecting expression from the internal cavity of the centrifugal chamber of a cell sample for culture, such as a cell sample containing cells transduced with a viral vector encoding the recombinant receptor, into the bioreactor bag of the provided assembly. In some embodiments, the bag is connected to a system containing the centrifugal chamber at an output line or output position, thereby resulting in transfer of the cells from the internal cavity of the chamber into the bioreactor bag for subsequent culturing or cultivation.

In some embodiments, the total volume of the cells and media transferred or filled into the bioreactor bag is from or from about 50 mL to 5000 mL, such as from or from about 300 mL to 3000 mL, 300 mL to 1500 mL, 300 mL to 1000 mL, 300 mL to 1000 mL, 300 mL to 500 mL, 500 mL to 3000 mL, 500 mL to 1500 mL, 500 mL to 1000 mL, 1000 mL to 2000 mL, 1000 mL to 1500 mL or 1500 mL to 2000 mL. In some embodiments, the total volume of the cells and media transferred or filled into the bioreactor bag is at least or about at least or about 50 mL, 100 mL, 300 mL, 500 mL, 750 mL, 1000 mL, 1250 mL, 1500 mL, 1750 mL or 2000 mL. In some embodiments, the bioreactor bag is capable of holding a total volume of from or from about 300 mL to 10 L, such as from or from about 500 mL to 5000 mL, 500 mL to 2500 mL, 500 mL to 2000 mL, 500 mL to 1500 mL, 500 mL to 1000 mL, 1000 mL to 5000 mL, 1000 mL to 2500 mL, 1000 mL to 2000 mL, 1000 mL to 1500 mL, 1500 mL to 5000 mL, 1500 mL to 2500 mL, 1500 mL to 2000 mL, 2000 mL to 5000 mL, 2000 mL to 2500 mL, or 2500 mL to 5000 mL.

In some aspects, the culture media is an adapted culture medium that supports that growth, cultivation, expansion or proliferation of the cells, such as T cells. In some aspects, the medium can be a liquid containing a mixture of salts, amino acids, vitamins, sugars or any combination thereof. In some embodiments, the culture media further contains one or more stimulating conditions or agents, such as to stimulate the cultivation, expansion or proliferation of cells during the incubation. In some embodiments, the stimulating condition is or includes one or more cytokine selected from IL-2, IL-7 or IL-15. In some embodiments, the cytokine is a recombinant cytokine. In some embodiments, the concentration of the one or more cytokine in the culture media during the culturing or incubation, independently, is from or from about 1 IU/mL to 1500 IU/mL, such as from or from about 1 IU/mL to 100 IU/mL, 2 IU/mL to 50 IU/mL, 5 IU/mL to 10 IU/mL, 10 IU/mL to 500 IU/mL, 50 IU/mL to 250 IU/mL or 100 IU/mL to 200 IU/mL, 50 IU/mL to 1500 IU/mL, 100 IU/mL to 1000 IU/mL or 200 IU/mL to 600 IU/mL. In some embodiments, the concentration of the one or more cytokine, independently, is at least or at least about 1 IU/mL, 5 IU/mL, 10 IU/mL, 50 IU/mL, 100 IU/mL, 200 IU/mL, 500 IU/mL, 1000 IU/mL or 1500 IU/mL. In certain aspects, an agent capable of activating an intracellular signaling domain of a TCR complex, such as an anti-CD3 and/or anti-CD28 antibody, also can be including during or during at least a portion of the incubating or subsequent to the incubating.

In some aspects, the bioreactor bag, such as a perfusion bag provided herein, is incubated for at least a portion of time after transfer of the cells and culture media. In some embodiments, the stimulating conditions generally include a temperature suitable for the growth of primary immune cells, such as human T lymphocytes, for example, at least about 25 degrees Celsius, generally at least about 30 degrees, and generally at or about 37 degrees Celsius. In some embodiments, the bioreactor bag is incubated at a temperature of 25 to 38° C., such as 30 to 37° C., for example at or about 37° C.±2° C. In some embodiments, the incubation is carried out for a time period until the culture, e.g. cultivation or expansion, results in a desired or threshold density, number or dose of cells. In some embodiments, the incubation is greater than or greater than about or is for about or 24 hours, 48 hours, 72 hours, 96 hours, 5 days, 6 days, 7 days, 8 days, 9 days or more.

In some embodiments, the bioreactor bag assembly is cultured under conditions to maintain a target amount of carbon dioxide in the cell culture. In some aspects, this ensures optimal cultivation, expansion and proliferation of the cells during the growth. In some aspects, the amount of carbon dioxide (CO₂) is between 10% and 0% (v/v) of said gas, such as between 8% and 2% (v/v) of said gas, for example an amount of or about 5% (v/v) CO₂.

In some cases, the bioreactor can be subject to motioning or rocking, which, in some aspects, can increase oxygen transfer. Motioning the bioreactor may include, but is not limited to rotating along a horizontal axis, rotating along a vertical axis, a rocking motion along a tilted or inclined horizontal axis of the bioreactor or any combination thereof. In some embodiments, at least a portion of the incubation is carried out with rocking. The rocking speed and rocking angle may be adjusted to achieve a desired agitation. In some embodiments the rock angle is 20°, 19°, 18°, 17°, 16°, 15°, 14°, 13°, 12°, 11°, 10°, 9°, 8°, 7°, 6°, 5°, 4°, 3°, 2° or 1°. In certain embodiments, the rock angle is between 6-16°. In other embodiments, the rock angle is between 7-16°. In other embodiments, the rock angle is between 8-12°. In some embodiments, the rock rate is 1, 2, 3, 4, 5, 6, 7, 8, 9, 10, 11, 1 12, 13, 14 15, 16, 17, 18, 19, 20, 21, 22, 23, 24, 25, 26, 27, 28, 29, 30, 31, 32, 33, 34, 35, 36, 37, 38, 39, 40 rpm. In some embodiments, the rock rate is between 4 and 12 rpm, such as between 4 and 6 rpm, inclusive.

In some embodiments, at least a portion of the incubation is carried out under static conditions. In some embodiments, at least a portion of the incubation is carried out with perfusion, such as to perfuse out spent media and perfuse in fresh media during the culture. In some embodiments, the method includes a step of perfusing fresh culture medium into the cell culture, such as through a feed port. In some embodiments, the culture media added during perfusion contains the one or more stimulating agents, e.g. one or more recombinant cytokine, such as IL-2, IL-7 and/or IL-15. In some embodiments, the culture media added during perfusion is the same culture media used during a static incubation.

In some embodiments, subsequent to the incubation, the bioreactor bag assembly is re-connected to a system for carrying out the one or more other processing steps of for manufacturing, generating or producing the cell therapy, such as is re-connected to the system containing the centrifugal chamber. In some aspects, cultured cells are transferred from the bioreactor bag to the internal cavity of the chamber for formulation of the cultured cells.

B. Other Processing Steps

In some embodiments, the culturing can be carried out in connection with one or more further processing steps, such as in connection with cell engineering. Such one or more processing steps can be carried out as part of the same closed system or in operable connection to the same closed system.

In some embodiments, the one or more processing steps include one or more of (a) washing a biological sample containing cells (e.g., a whole blood sample, a buffy coat sample, a peripheral blood mononuclear cells (PBMC) sample, an unfractionated T cell sample, a lymphocyte sample, a white blood cell sample, an apheresis product, or a leukapheresis product), (b) isolating, e.g. selecting, from the sample a desired subset or population of cells (e.g., CD4+ and/or CD8+ T cells), for example, by incubation of cells with a selection or immunoaffinity reagent for immunoaffinity-based separation; c) incubating the isolated, such as selected cells, with viral vector particles, (d) culturing, cultivating or expanding the cells such as in accord with the methods described above and (e) formulating the transduced cells, such as in a pharmaceutically acceptable buffer, cryopreservative or other suitable medium. In some embodiments, the methods can further include (e) stimulating cells by exposing cells to stimulating conditions, which can be performed prior to, during and/or subsequent to the incubation of cells with viral vector particles. In some embodiments, one or more further step of washing or suspending step, such as for dilution, concentration and/or buffer exchange of cells, can also be carried out prior to or subsequent to any of the above steps.

1. Isolation or Selection of Cells from Samples

In some embodiments, the processing steps include isolation of cells or compositions thereof from biological samples, such as those obtained from or derived from a subject, such as one having a particular disease or condition or in need of a cell therapy or to which cell therapy will be administered. In some aspects, the subject is a human, such as a subject who is a patient in need of a particular therapeutic intervention, such as the adoptive cell therapy for which cells are being isolated, processed, and/or engineered. Accordingly, the cells in some embodiments are primary cells, e.g., primary human cells. The samples include tissue, fluid, and other samples taken directly from the subject. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media.

In some embodiments, the preparation methods include steps for freezing, e.g., cryopreserving, the cells, either before or after isolation, selection and/or enrichment and/or incubation for transduction and engineering. In some embodiments, the freeze and subsequent thaw step removes granulocytes and, to some extent, monocytes in the cell population. In some embodiments, the cells are suspended in a freezing solution, e.g., following a washing step to remove plasma and platelets. Any of a variety of known freezing solutions and parameters in some aspects may be used. One example involves using PBS containing 20% DMSO and 8% human serum albumin (HSA), or other suitable cell freezing media. This is then diluted 1:1 with media so that the final concentration of DMSO and HSA are 10% and 4%, respectively. The cells are generally then frozen to −80° C. at a rate of 1° per minute and stored in the vapor phase of a liquid nitrogen storage tank.

In some embodiments, isolation of the cells or populations includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components. In some embodiments, the methods include density-based cell separation methods, such as the preparation of white blood cells from peripheral blood by lysing the red blood cells and centrifugation through a Percoll or Ficoll gradient.

In some embodiments, at least a portion of the selection step includes incubation of cells with a selection reagent. The incubation with a selection reagent or reagents, e.g., as part of selection methods which may be performed using one or more selection reagents for selection of one or more different cell types based on the expression or presence in or on the cell of one or more specific molecules, such as surface markers, e.g., surface proteins, intracellular markers, or nucleic acid. In some embodiments, any known method using a selection reagent or reagents for separation based on such markers may be used. In some embodiments, the selection reagent or reagents result in a separation that is affinity- or immunoaffinity-based separation. For example, the selection in some aspects includes incubation with a reagent or reagents for separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

In some aspects of such processes, a volume of cells is mixed with an amount of a desired affinity-based selection reagent. The immunoaffinity-based selection can be carried out using any system or method that results in a favorable energetic interaction between the cells being separated and the molecule specifically binding to the marker on the cell, e.g., the antibody or other binding partner on the solid surface, e.g., particle. In some embodiments, methods are carried out using particles such as beads, e.g. magnetic beads, that are coated with a selection agent (e.g. antibody) specific to the marker of the cells. The particles (e.g. beads) can be incubated or mixed with cells in a container, such as a tube or bag, while shaking or mixing, with a constant cell density-to-particle (e.g., bead) ratio to aid in promoting energetically favored interactions. In other cases, the methods include selection of cells in which all or a portion of the selection is carried out in the internal cavity of a centrifugal chamber, for example, under centrifugal rotation. In some embodiments, incubation of cells with selection reagents, such as immunoaffinity-based selection reagents, is performed in a centrifugal chamber. In certain embodiments, the isolation or separation is carried out using a system, device, or apparatus described in International Patent Application, Publication Number WO2009/072003, or US 20110003380 A1. In one example, the system is a system as described in International Publication Number WO2016/073602.

In some embodiments, by conducting such selection steps or portions thereof (e.g., incubation with antibody-coated particles, e.g., magnetic beads) in the cavity of a centrifugal chamber, the user is able to control certain parameters, such as volume of various solutions, addition of solution during processing and timing thereof, which can provide advantages compared to other available methods. For example, the ability to decrease the liquid volume in the cavity during the incubation can increase the concentration of the particles (e.g. bead reagent) used in the selection, and thus the chemical potential of the solution, without affecting the total number of cells in the cavity. This in turn can enhance the pairwise interactions between the cells being processed and the particles used for selection. In some embodiments, carrying out the incubation step in the chamber, e.g., when associated with the systems, circuitry, and control as described herein, permits the user to effect agitation of the solution at desired time(s) during the incubation, which also can improve the interaction.

In some embodiments, at least a portion of the selection step is performed in a centrifugal chamber, which includes incubation of cells with a selection reagent. In some aspects of such processes, a volume of cells is mixed with an amount of a desired affinity-based selection reagent that is far less than is normally employed when performing similar selections in a tube or container for selection of the same number of cells and/or volume of cells according to manufacturer's instructions. In some embodiments, an amount of selection reagent or reagents that is/are no more than 5%, no more than 10%, no more than 15%, no more than 20%, no more than 25%, no more than 50%, no more than 60%, no more than 70% or no more than 80% of the amount of the same selection reagent(s) employed for selection of cells in a tube or container-based incubation for the same number of cells and/or the same volume of cells according to manufacturer's instructions is employed.

In some embodiments, for selection, e.g., immunoaffinity-based selection of the cells, the cells are incubated in the cavity of the chamber in a composition that also contains the selection buffer with a selection reagent, such as a molecule that specifically binds to a surface marker on a cell that it desired to enrich and/or deplete, but not on other cells in the composition, such as an antibody, which optionally is coupled to a scaffold such as a polymer or surface, e.g., bead, e.g., magnetic bead, such as magnetic beads coupled to monoclonal antibodies specific for CD4 and CD8. In some embodiments, as described, the selection reagent is added to cells in the cavity of the chamber in an amount that is substantially less than (e.g. is no more than 5%, 10%, 20%, 30%, 40%, 50%, 60%, 70% or 80% of the amount) as compared to the amount of the selection reagent that is typically used or would be necessary to achieve about the same or similar efficiency of selection of the same number of cells or the same volume of cells when selection is performed in a tube with shaking or rotation. In some embodiments, the incubation is performed with the addition of a selection buffer to the cells and selection reagent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the selection buffer and selection reagent are pre-mixed before addition to the cells. In some embodiments, the selection buffer and selection reagent are separately added to the cells. In some embodiments, the selection incubation is carried out with periodic gentle mixing condition, which can aid in promoting energetically favored interactions and thereby permit the use of less overall selection reagent while achieving a high selection efficiency.

In some embodiments, the total duration of the incubation with the selection reagent is from or from about 5 minutes to 6 hours, such as 30 minutes to 3 hours, for example, at least or about at least 30 minutes, 60 minutes, 120 minutes or 180 minutes.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, such process is carried out within the entirely closed system to which the chamber is integral. In some embodiments, this process (and in some aspects also one or more additional step, such as a previous wash step washing a sample containing the cells, such as an apheresis sample) is carried out in an automated fashion, such that the cells, reagent, and other components are drawn into and pushed out of the chamber at appropriate times and centrifugation effected, so as to complete the wash and binding step in a single closed system using an automated program.

In some embodiments, after the incubation and/or mixing of the cells and selection reagent and/or reagents, the incubated cells are subjected to a separation to select for cells based on the presence or absence of the particular reagent or reagents. In some embodiments, the separation is performed in the same closed system in which the incubation of cells with the selection reagent was performed. In some embodiments, after incubation with the selection reagents, incubated cells, including cells in which the selection reagent has bound are transferred into a system for immunoaffinity-based separation of the cells. In some embodiments, the system for immunoaffinity-based separation is or contains a magnetic separation column.

Such separation steps can be based on positive selection, in which the cells having bound the reagents, e.g. antibody or binding partner, are retained for further use, and/or negative selection, in which the cells having not bound to the reagent, e.g., antibody or binding partner, are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

In some embodiments, the process steps further include negative and/or positive selection of the incubated and cells, such as using a system or apparatus that can perform an affinity-based selection. In some embodiments, isolation is carried out by enrichment for a particular cell population by positive selection, or depletion of a particular cell population, by negative selection. In some embodiments, positive or negative selection is accomplished by incubating cells with one or more antibodies or other binding agent that specifically bind to one or more surface markers expressed or expressed (marker+) at a relatively higher level (marker^{high}) on the positively or negatively selected cells, respectively.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

For example, in some aspects, specific subpopulations of T cells, such as cells positive or expressing high levels of one or more surface markers, e.g., CD28+, CD62L+, CCR7+, CD27+, CD127+, CD4+, CD8+, CD45RA+, and/or CD45RO+ T cells, are isolated by positive or negative selection techniques. In some embodiments, such cells are selected by incubation with one or more antibody or binding partner that specifically binds to such markers. In some embodiments, the antibody or binding partner can be conjugated, such as directly or indirectly, to a solid support or matrix to effect selection, such as a magnetic bead or paramagnetic bead. For example, CD3+, CD28+ T cells can be positively selected using CD3/CD28 conjugated magnetic beads (e.g., DYNABEADS® M-450 CD3/CD28 T Cell Expander, and/or ExpACT® beads).

In some embodiments, T cells are separated from a PBMC sample by negative selection of markers expressed on non-T cells, such as B cells, monocytes, or other white blood cells, such as CD14. In some aspects, a CD4+ or CD8+ selection step is used to separate CD4+ helper and CD8+ cytotoxic T cells. Such CD4+ and CD8+ populations can be further sorted into sub-populations by positive or negative selection for markers expressed or expressed to a relatively higher degree on one or more naive, memory, and/or effector T cell subpopulations.

In some embodiments, CD8+ cells are further enriched for or depleted of naive, central memory, effector memory, and/or central memory stem cells, such as by positive or negative selection based on surface antigens associated with the respective subpopulation. In some embodiments, enrichment for central memory T (TCM) cells is carried out to increase efficacy, such as to improve long-term survival, expansion, and/or engraftment following administration, which in some aspects is particularly robust in such sub-populations. See Terakura et al., (2012) Blood. 1:72-82; Wang et al. (2012) J Immunother. 35(9):689-701. In some embodiments, combining TCM-enriched CD8+ T cells and CD4+ T cells further enhances efficacy.

In embodiments, memory T cells are present in both CD62L+ and CD62L-subsets of CD8+ peripheral blood lymphocytes. PBMC can be enriched for or depleted of CD62L-CD8+ and/or CD62L+CD8+ fractions, such as using anti-CD8 and anti-CD62L antibodies.

In some embodiments, the enrichment for central memory T (TCM) cells is based on positive or high surface expression of CD45RO, CD62L, CCR7, CD28, CD3, and/or CD 127; in some aspects, it is based on negative selection for cells expressing or highly expressing CD45RA and/or granzyme B. In some aspects, isolation of a CD8+ population enriched for TCM cells is carried out by depletion of cells expressing CD4, CD14, CD45RA, and positive selection or enrichment for cells expressing CD62L. In one aspect, enrichment for central memory T (TCM) cells is carried out starting with a negative fraction of cells selected based on CD4 expression, which is subjected to a negative selection based on expression of CD14 and CD45RA, and a positive selection based on CD62L. Such selections in some aspects are carried out simultaneously and in other aspects are carried out sequentially, in either order. In some aspects, the same CD4 expression-based selection step used in preparing the CD8+ cell population or subpopulation, also is used to generate the CD4+ cell population or sub-population, such that both the positive and negative fractions from the CD4-based separation are retained and used in subsequent steps of the methods, optionally following one or more further positive or negative selection steps. In some embodiments, the selection for the CD4+ cell population and the selection for the CD8+ cell population are carried out simultaneously. In some embodiments, the CD4+ cell population and the selection for the CD8+ cell population are carried out sequentially, in either order. In some embodiments, methods for selecting cells can include those as described in published U.S. App. No. US20170037369. In some embodiments, the selected CD4+ cell population and the selected CD8+ cell population may be combined subsequent to the selecting. In some aspects, the selected CD4+ cell population and the selected CD8+ cell population may be combined in a bioreactor bag as described herein.

In a particular example, a sample of PBMCs or other white blood cell sample is subjected to selection of CD4+ cells, where both the negative and positive fractions are retained. The negative fraction then is subjected to negative selection based on expression of CD14 and CD45RA or CD19, and positive selection based on a marker characteristic of central memory T cells, such as CD62L or CCR7, where the positive and negative selections are carried out in either order.

CD4+ T helper cells may be sorted into naïve, central memory, and effector cells by identifying cell populations that have cell surface antigens. CD4+ lymphocytes can be obtained by standard methods. In some embodiments, naive CD4+ T lymphocytes are CD45RO−, CD45RA+, CD62L+, or CD4+ T cells. In some embodiments, central memory CD4+ cells are CD62L+ and CD45RO+. In some embodiments, effector CD4+ cells are CD62L− and CD45RO−.

In one example, to enrich for CD4+ cells by negative selection, a monoclonal antibody cocktail typically includes antibodies to CD14, CD20, CD11b, CD16, HLA-DR, and CD8. In some embodiments, the antibody or binding partner is bound to a solid support or matrix, such as a magnetic bead or paramagnetic bead, to allow for separation of cells for positive and/or negative selection. For example, in some embodiments, the cells and cell populations are separated or isolated using immunomagnetic (or affinitymagnetic) separation techniques (reviewed in Methods in Molecular Medicine, vol. 58: Metastasis Research Protocols, Vol. 2: Cell Behavior In Vitro and In Vivo, p 17-25 Edited by: S. A. Brooks and U. Schumacher © Humana Press Inc., Totowa, N.J.).

In some aspects, the incubated sample or composition of cells to be separated is incubated with a selection reagent containing small, magnetizable or magnetically responsive material, such as magnetically responsive particles or microparticles, such as paramagnetic beads (e.g., such as Dynalbeads or MACS® beads). The magnetically responsive material, e.g., particle, generally is directly or indirectly attached to a binding partner, e.g., an antibody, that specifically binds to a molecule, e.g., surface marker, present on the cell, cells, or population of cells that it is desired to separate, e.g., that it is desired to negatively or positively select.

In some embodiments, the magnetic particle or bead comprises a magnetically responsive material bound to a specific binding member, such as an antibody or other binding partner. Many well-known magnetically responsive materials for use in magnetic separation methods are known, e.g., those described in Molday, U.S. Pat. No. 4,452,773, and in European Patent Specification EP 452342 B, which are hereby incorporated by reference. Colloidal sized particles, such as those described in Owen U.S. Pat. No. 4,795,698, and Liberti et al., U.S. Pat. No. 5,200,084 also may be used.

The incubation generally is carried out under conditions whereby the antibodies or binding partners, or molecules, such as secondary antibodies or other reagents, which specifically bind to such antibodies or binding partners, which are attached to the magnetic particle or bead, specifically bind to cell surface molecules if present on cells within the sample.

In certain embodiments, the magnetically responsive particles are coated in primary antibodies or other binding partners, secondary antibodies, lectins, enzymes, or streptavidin. In certain embodiments, the magnetic particles are attached to cells via a coating of primary antibodies specific for one or more markers. In certain embodiments, the cells, rather than the beads, are labeled with a primary antibody or binding partner, and then cell-type specific secondary antibody- or other binding partner (e.g., streptavidin)-coated magnetic particles, are added. In certain embodiments, streptavidin-coated magnetic particles are used in conjunction with biotinylated primary or secondary antibodies.

In some aspects, separation is achieved in a procedure in which the sample is placed in a magnetic field, and those cells having magnetically responsive or magnetizable particles attached thereto will be attracted to the magnet and separated from the unlabeled cells. For positive selection, cells that are attracted to the magnet are retained; for negative selection, cells that are not attracted (unlabeled cells) are retained. In some aspects, a combination of positive and negative selection is performed during the same selection step, where the positive and negative fractions are retained and further processed or subject to further separation steps.

In some embodiments, the affinity-based selection is via magnetic-activated cell sorting (MACS) (Miltenyi Biotech, Auburn, Calif.). Magnetic Activated Cell Sorting (MACS), e.g., CliniMACS systems are capable of high-purity selection of cells having magnetized particles attached thereto. In certain embodiments, MACS operates in a mode wherein the non-target and target species are sequentially eluted after the application of the external magnetic field. That is, the cells attached to magnetized particles are held in place while the unattached species are eluted. Then, after this first elution step is completed, the species that were trapped in the magnetic field and were prevented from being eluted are freed in some manner such that they can be eluted and recovered. In certain embodiments, the non-target cells are labelled and depleted from the heterogeneous population of cells.

In some embodiments, the magnetically responsive particles are left attached to the cells that are to be subsequently incubated, cultured and/or engineered; in some aspects, the particles are left attached to the cells for administration to a patient. In some embodiments, the magnetizable or magnetically responsive particles are removed from the cells. Methods for removing magnetizable particles from cells are known and include, e.g., the use of competing non-labeled antibodies, magnetizable particles or antibodies conjugated to cleavable linkers, etc. In some embodiments, the magnetizable particles are biodegradable.

2. Genetic Engineering

In some embodiments, the processing steps include introduction of a nucleic acid molecule encoding a recombinant protein. Exemplary of such recombinant proteins are recombinant receptors, such as any described in Section III. Introduction of the nucleic acid molecules encoding the recombinant protein, such as recombinant receptor, in the cell may be carried out using any of a number of known vectors. Such vectors include viral and non-viral systems, including lentiviral and gammaretroviral systems, as well as transposon-based systems such as PiggyBac or Sleeping Beauty-based gene transfer systems. Exemplary methods include those for transfer of nucleic acids encoding the receptors, including via viral, e.g., retroviral or lentiviral, transduction, transposons, and electroporation.

In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical applications.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors (see, e.g., Koste et al. (2014) Gene Therapy 2014 Apr. 3. doi: 10.1038/gt.2014.25; Carlens et al. (2000) Exp Hematol 28(10): 1137-46; Alonso-Camino et al. (2013) Mol Ther Nucl Acids 2, e93; Park et al., Trends Biotechnol. 2011 Nov. 29(11): 550-557.

In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), spleen focus forming virus (SFFV), or adeno-associated virus (AAV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In one embodiment, the gene to be expressed replaces the retroviral gag, pol and/or env sequences. A number of illustrative retroviral systems have been described (e.g., U.S. Pat. Nos. 5,219,740; 6,207,453; 5,219,740; Miller and Rosman (1989) BioTechniques 7:980-990; Miller, A. D. (1990) Human Gene Therapy 1:5-14; Scarpa et al. (1991) Virology 180:849-852; Burns et al. (1993) Proc. Natl. Acad. Sci. USA 90:8033-8037; and Boris-Lawrie and Temin (1993) Cur. Opin. Genet. Develop. 3:102-109.

Methods of lentiviral transduction are known. Exemplary methods are described in, e.g., Wang et al. (2012) J. Immunother. 35(9): 689-701; Cooper et al. (2003) Blood. 101:1637-1644; Verhoeyen et al. (2009) Methods Mol Biol. 506: 97-114; and Cavalieri et al. (2003) Blood. 102(2): 497-505.

In some embodiments, recombinant nucleic acids are transferred into T cells via electroporation (see, e.g., Chicaybam et al, (2013) PLoS ONE 8(3): e60298 and Van Tedeloo et al. (2000) Gene Therapy 7(16): 1431-1437). In some embodiments, recombinant nucleic acids are transferred into T cells via transposition (see, e.g., Manuri et al. (2010) Hum Gene Ther 21(4): 427-437; Sharma et al. (2013) Molec Ther Nucl Acids 2, e74; and Huang et al. (2009) Methods Mol Biol 506: 115-126). Other methods of introducing and expressing genetic material in immune cells include calcium phosphate transfection (e.g., as described in Current Protocols in Molecular Biology, John Wiley & Sons, New York. N.Y.), protoplast fusion, cationic liposome-mediated transfection; tungsten particle-facilitated microparticle bombardment (Johnston, Nature, 346: 776-777 (1990)); and strontium phosphate DNA co-precipitation (Brash et al., Mol. Cell Biol., 7: 2031-2034 (1987)).

Other approaches and vectors for transfer of the nucleic acids encoding the recombinant products are those described, e.g., in international patent application, Publication No.: WO2014055668, and U.S. Pat. No. 7,446,190.

In some embodiments, the cells, e.g., T cells, may be transfected either during or after expansion e.g. with a T cell receptor (TCR) or a chimeric antigen receptor (CAR). This transfection for the introduction of the gene of the desired receptor can be carried out with any suitable retroviral vector, for example. The genetically modified cell population can then be liberated from the initial stimulus (the CD3/CD28 stimulus, for example) and subsequently be stimulated with a second type of stimulus e.g. via a de novo introduced receptor). This second type of stimulus may include an antigenic stimulus in form of a peptide/MHC molecule, the cognate (cross-linking) ligand of the genetically introduced receptor (e.g. natural ligand of a CAR) or any ligand (such as an antibody) that directly binds within the framework of the new receptor (e.g. by recognizing constant regions within the receptor). See, for example, Cheadle et al, “Chimeric antigen receptors for T-cell based therapy” Methods Mol Biol. 2012; 907:645-66 or Barrett et al., Chimeric Antigen Receptor Therapy for Cancer Annual Review of Medicine Vol. 65: 333-347 (2014).

In some cases, a vector may be used that does not require that the cells, e.g., T cells, are activated. In some such instances, the cells may be selected and/or transduced prior to activation. Thus, the cells may be engineered prior to, or subsequent to culturing of the cells, and in some cases at the same time as or during at least a portion of the culturing.

In some aspects, the cells further are engineered to promote expression of cytokines or other factors. Among additional nucleic acids, e.g., genes for introduction are those to improve the efficacy of therapy, such as by promoting viability and/or function of transferred cells; genes to provide a genetic marker for selection and/or evaluation of the cells, such as to assess in vivo survival or localization; genes to improve safety, for example, by making the cell susceptible to negative selection in vivo as described by Lupton S. D. et al., Mol. and Cell Biol., 11:6 (1991); and Riddell et al., Human Gene Therapy 3:319-338 (1992); see also the publications of PCT/US91/08442 and PCT/US94/05601 by Lupton et al. describing the use of bifunctional selectable fusion genes derived from fusing a dominant positive selectable marker with a negative selectable marker. See, e.g., Riddell et al., U.S. Pat. No. 6,040,177, at columns 14-17.

In some embodiments, the introducing is carried out by contacting one or more cells of a composition with a nucleic acid molecule encoding the recombinant protein, e.g. recombinant receptor. In some embodiments, the contacting can be effected with centrifugation, such as spinoculation (e.g. centrifugal inoculation). Such methods include any of those as described in International Publication Number WO2016/073602. Exemplary centrifugal chambers include those produced and sold by Biosafe SA, including those for use with the Sepax® and Sepax® 2 system, including an A-200/F and A-200 centrifugal chambers and various kits for use with such systems. Exemplary chambers, systems, and processing instrumentation and cabinets are described, for example, in U.S. Pat. Nos. 6,123,655, 6,733,433 and Published U.S. Patent Application, Publication No.: US 2008/0171951, and published international patent application, publication no. WO 00/38762, the contents of each of which are incorporated herein by reference in their entirety. Exemplary kits for use with such systems include, but are not limited to, single-use kits sold by BioSafe SA under product names CS-430.1, CS-490.1, CS-600.1 or CS-900.2.

In some embodiments, the system is included with and/or placed into association with other instrumentation, including instrumentation to operate, automate, control and/or monitor aspects of the transduction step and one or more various other processing steps performed in the system, e.g. one or more processing steps that can be carried out with or in connection with the centrifugal chamber system as described herein or in International Publication Number WO2016/073602. This instrumentation in some embodiments is contained within a cabinet. In some embodiments, the instrumentation includes a cabinet, which includes a housing containing control circuitry, a centrifuge, a cover, motors, pumps, sensors, displays, and a user interface. An exemplary device is described in U.S. Pat. Nos. 6,123,655, 6,733,433 and US 2008/0171951.

In some embodiments, the system comprises a series of containers, e.g., bags, tubing, stopcocks, clamps, connectors, and a centrifuge chamber. In some embodiments, the containers, such as bags, include one or more containers, such as bags, containing the cells to be transduced and the viral vector particles, in the same container or separate containers, such as the same bag or separate bags. In some embodiments, the system further includes one or more containers, such as bags, containing medium, such as diluent and/or wash solution, which is pulled into the chamber and/or other components to dilute, resuspend, and/or wash components and/or compositions during the methods. The containers can be connected at one or more positions in the system, such as at a position corresponding to an input line, diluent line, wash line, waste line and/or output line.

In some embodiments, the chamber is associated with a centrifuge, which is capable of effecting rotation of the chamber, such as around its axis of rotation. Rotation may occur before, during, and/or after the incubation in connection with transduction of the cells and/or in one or more of the other processing steps. Thus, in some embodiments, one or more of the various processing steps is carried out under rotation, e.g., at a particular force. The chamber is typically capable of vertical or generally vertical rotation, such that the chamber sits vertically during centrifugation and the side wall and axis are vertical or generally vertical, with the end wall(s) horizontal or generally horizontal.

In some embodiments, the composition containing cells, viral particles and reagent can be rotated, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm). In some embodiments, the rotation is carried at a force, e.g., a relative centrifugal force, of from or from about 100 g to 3200 g (e.g. at or about or at least at or about 100 g, 200 g, 300 g, 400 g, 500 g, 1000 g, 1500 g, 2000 g, 2500 g, 3000 g or 3200 g), as measured for example at an internal or external wall of the chamber or cavity. The term “relative centrifugal force” or RCF is generally understood to be the effective force imparted on an object or substance (such as a cell, sample, or pellet and/or a point in the chamber or other container being rotated), relative to the earth's gravitational force, at a particular point in space as compared to the axis of rotation. The value may be determined using well-known formulas, taking into account the gravitational force, rotation speed and the radius of rotation (distance from the axis of rotation and the object, substance, or particle at which RCF is being measured).

In some embodiments, during at least a part of the genetic engineering, e.g. transduction, and/or subsequent to the genetic engineering the cells are transferred to the bioreactor bag assembly for culture of the genetically engineered cells, such as for cultivation or expansion of the cells, as described above.

Preparation of Viral Vector Particles for Transduction

The viral vector genome is typically constructed in a plasmid form that can be transfected into a packaging or producer cell line. In any of such examples, the nucleic acid encoding a recombinant protein, such as a recombinant receptor, is inserted or located in a region of the viral vector, such as generally in a non-essential region of the viral genome. In some embodiments, the nucleic acid is inserted into the viral genome in the place of certain viral sequences to produce a virus that is replication defective.

Any of a variety of known methods can be used to produce retroviral particles whose genome contains an RNA copy of the viral vector genome. In some embodiments, at least two components are involved in making a virus-based gene delivery system: first, packaging plasmids, encompassing the structural proteins as well as the enzymes necessary to generate a viral vector particle, and second, the viral vector itself, i.e., the genetic material to be transferred. Biosafety safeguards can be introduced in the design of one or both of these components.

In some embodiments, the packaging plasmid can contain all retroviral, such as HIV-1, proteins other than envelope proteins (Naldini et al., 1998). In other embodiments, viral vectors can lack additional viral genes, such as those that are associated with virulence, e.g. vpr, vif, vpu and nef, and/or Tat, a primary transactivator of HIV. In some embodiments, lentiviral vectors, such as HIV-based lentiviral vectors, comprise only three genes of the parental virus: gag, pol and rev, which reduces or eliminates the possibility of reconstitution of a wild-type virus through recombination.

In some embodiments, the viral vector genome is introduced into a packaging cell line that contains all the components necessary to package viral genomic RNA, transcribed from the viral vector genome, into viral particles. Alternatively, the viral vector genome may comprise one or more genes encoding viral components in addition to the one or more sequences, e.g., recombinant nucleic acids, of interest. In some aspects, in order to prevent replication of the genome in the target cell, however, endogenous viral genes required for replication are removed and provided separately in the packaging cell line.

In some embodiments, a packaging cell line is transfected with one or more plasmid vectors containing the components necessary to generate the particles. In some embodiments, a packaging cell line is transfected with a plasmid containing the viral vector genome, including the LTRs, the cis-acting packaging sequence and the sequence of interest, i.e. a nucleic acid encoding an antigen receptor, such as a CAR; and one or more helper plasmids encoding the virus enzymatic and/or structural components, such as Gag, pol and/or rev. In some embodiments, multiple vectors are utilized to separate the various genetic components that generate the retroviral vector particles. In some such embodiments, providing separate vectors to the packaging cell reduces the chance of recombination events that might otherwise generate replication competent viruses. In some embodiments, a single plasmid vector having all of the retroviral components can be used.

In some embodiments, the retroviral vector particle, such as lentiviral vector particle, is pseudotyped to increase the transduction efficiency of host cells. For example, a retroviral vector particle, such as a lentiviral vector particle, in some embodiments is pseudotyped with a VSV-G glycoprotein, which provides a broad cell host range extending the cell types that can be transduced. In some embodiments, a packaging cell line is transfected with a plasmid or polynucleotide encoding a non-native envelope glycoprotein, such as to include xenotropic, polytropic or amphotropic envelopes, such as Sindbis virus envelope, GALV or VSV-G.

In some embodiments, the packaging cell line provides the components, including viral regulatory and structural proteins, that are required in trans for the packaging of the viral genomic RNA into lentiviral vector particles. In some embodiments, the packaging cell line may be any cell line that is capable of expressing lentiviral proteins and producing functional lentiviral vector particles. In some aspects, suitable packaging cell lines include 293 (ATCC CCL X), 293T, HeLA (ATCC CCL 2), D17 (ATCC CCL 183), MDCK (ATCC CCL 34), BHK (ATCC CCL-10) and Cf2Th (ATCC CRL 1430) cells.

In some embodiments, the packaging cell line stably expresses the viral protein(s). For example, in some aspects, a packaging cell line containing the gag, pol, rev and/or other structural genes but without the LTR and packaging components can be constructed. In some embodiments, a packaging cell line can be transiently transfected with nucleic acid molecules encoding one or more viral proteins along with the viral vector genome containing a nucleic acid molecule encoding a heterologous protein, and/or a nucleic acid encoding an envelope glycoprotein.

In some embodiments, the viral vectors and the packaging and/or helper plasmids are introduced via transfection or infection into the packaging cell line. The packaging cell line produces viral vector particles that contain the viral vector genome. Methods for transfection or infection are well known. Non-limiting examples include calcium phosphate, DEAE-dextran and lipofection methods, electroporation and microinjection.

When a recombinant plasmid and the retroviral LTR and packaging sequences are introduced into a special cell line (e.g., by calcium phosphate precipitation for example), the packaging sequences may permit the RNA transcript of the recombinant plasmid to be packaged into viral particles, which then may be secreted into the culture media. The media containing the recombinant retroviruses in some embodiments is then collected, optionally concentrated, and used for gene transfer. For example, in some aspects, after cotransfection of the packaging plasmids and the transfer vector to the packaging cell line, the viral vector particles are recovered from the culture media and titered by standard methods used by those of skill in the art.

In some embodiments, a retroviral vector, such as a lentiviral vector, can be produced in a packaging cell line, such as an exemplary HEK 293T cell line, by introduction of plasmids to allow generation of lentiviral particles. In some embodiments, a packaging cell is transfected and/or contains a polynucleotide encoding gag and pol, and a polynucleotide encoding a recombinant receptor, such as an antigen receptor, for example, a CAR. In some embodiments, the packaging cell line is optionally and/or additionally transfected with and/or contains a polynucleotide encoding a rev protein. In some embodiments, the packaging cell line is optionally and/or additionally transfected with and/or contains a polynucleotide encoding a non-native envelope glycoprotein, such as VSV-G. In some such embodiments, approximately two days after transfection of cells, e.g. HEK 293T cells, the cell supernatant contains recombinant lentiviral vectors, which can be recovered and titered.

Recovered and/or produced retroviral vector particles can be used to transduce target cells using the methods as described. Once in the target cells, the viral RNA is reverse-transcribed, imported into the nucleus and stably integrated into the host genome. One or two days after the integration of the viral RNA, the expression of the recombinant protein, e.g. antigen receptor, such as CAR, can be detected.

3. Activation and Stimulation

In some embodiments, the one or more processing steps include a step of stimulating the isolated cells, such as selected cell populations. The incubation may be prior to or in connection with genetic engineering, such as genetic engineering resulting from embodiments of the transduction method described above. In some embodiments, the stimulation results in activation and/or proliferation of the cells, for example, prior to transduction.

In some embodiments, the processing steps include incubations of cells, such as selected cells, in which the incubation steps can include culture, cultivation, stimulation, activation, and/or propagation of cells. In some embodiments, the compositions or cells are incubated in the presence of stimulating conditions or a stimulatory agent. Such conditions include those designed to induce proliferation, expansion, activation, and/or survival of cells in the population, to mimic antigen exposure, and/or to prime the cells for genetic engineering, such as for the introduction of a recombinant antigen receptor.

In some embodiments, the conditions for stimulation and/or activation can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some embodiments, the stimulating conditions or agents include one or more agent, e.g., ligand, which is capable of activating an intracellular signaling domain of a TCR complex. In some aspects, the agent turns on or initiates TCR/CD3 intracellular signaling cascade in a T cell, such as agents suitable to deliver a primary signal, e.g., to initiate activation of an ITAM-induced signal, such as those specific for a TCR component, and/or an agent that promotes a costimulatory signal, such as one specific for a T cell costimulatory receptor, e.g., anti-CD3, anti-CD28, or anti-41-BB, for example, bound to solid support such as a bead, and/or one or more cytokines. Among the stimulating agents are anti-CD3/anti-CD28 beads (e.g., DYNABEADS® M-450 CD3/CD28 T Cell Expander, and/or ExpACT® beads). Optionally, the expansion method may further comprise the step of adding anti-CD3 and/or anti CD28 antibody to the culture medium. In some embodiments, the stimulating agents include IL-2, IL-7 and/or IL-15, for example, an IL-2 concentration of at least about 10 units/mL, at least about 50 units/mL, at least about 100 units/mL or at least about 200 units/mL.

The conditions can include one or more of particular media, temperature, oxygen content, carbon dioxide content, time, agents, e.g., nutrients, amino acids, antibiotics, ions, and/or stimulatory factors, such as cytokines, chemokines, antigens, binding partners, fusion proteins, recombinant soluble receptors, and any other agents designed to activate the cells.

In some aspects, incubation is carried out in accordance with techniques such as those described in U.S. Pat. No. 6,040,177 to Riddell et al., Klebanoff et al. (2012) J Immunother. 35(9): 651-660, Terakura et al. (2012) Blood. 1:72-82, and/or Wang et al. (2012) J Immunother. 35(9):689-701.

In some embodiments, at least a portion of the incubation in the presence of one or more stimulating conditions or a stimulatory agents is carried out in the internal cavity of a centrifugal chamber, for example, under centrifugal rotation, such as described in International Publication Number WO2016/073602. In some embodiments, at least a portion of the incubation performed in a centrifugal chamber includes mixing with a reagent or reagents to induce stimulation and/or activation. In some embodiments, cells, such as selected cells, are mixed with a stimulating condition or stimulatory agent in the centrifugal chamber. In some aspects of such processes, a volume of cells is mixed with an amount of one or more stimulating conditions or agents that is far less than is normally employed when performing similar stimulations in a cell culture plate or other system.

In some embodiments, the stimulating agent is added to cells in the cavity of the chamber in an amount that is substantially less than (e.g. is no more than 5%, 10%, 20%, 30%, 40%, 50%, 60%, 70% or 80% of the amount) as compared to the amount of the stimulating agent that is typically used or would be necessary to achieve about the same or similar efficiency of selection of the same number of cells or the same volume of cells when selection is performed without mixing in a centrifugal chamber, e.g. in a tube or bag with periodic shaking or rotation. In some embodiments, the incubation is performed with the addition of an incubation buffer to the cells and stimulating agent to achieve a target volume with incubation of the reagent of, for example, 10 mL to 200 mL, such as at least or about at least or about or 10 mL, 20 mL, 30 mL, 40 mL, 50 mL, 60 mL, 70 mL, 80 mL, 90 mL, 100 mL, 150 mL or 200 mL. In some embodiments, the incubation buffer and stimulating agent are pre-mixed before addition to the cells. In some embodiments, the incubation buffer and stimulating agent are separately added to the cells. In some embodiments, the stimulating incubation is carried out with periodic gentle mixing condition, which can aid in promoting energetically favored interactions and thereby permit the use of less overall stimulating agent while achieving stimulating and activation of cells.

In some embodiments, the incubation generally is carried out under mixing conditions, such as in the presence of spinning, generally at relatively low force or speed, such as speed lower than that used to pellet the cells, such as from or from about 600 rpm to 1700 rpm (e.g. at or about or at least 600 rpm, 1000 rpm, or 1500 rpm or 1700 rpm), such as at an RCF at the sample or wall of the chamber or other container of from or from about 80 g to 100 g (e.g. at or about or at least 80 g, 85 g, 90 g, 95 g, or 100 g). In some embodiments, the spin is carried out using repeated intervals of a spin at such low speed followed by a rest period, such as a spin and/or rest for 1, 2, 3, 4, 5, 6, 7, 8, 9, or 10 seconds, such as a spin at approximately 1 or 2 seconds followed by a rest for approximately 5, 6, 7, or 8 seconds.

In some embodiments, the total duration of the incubation, e.g. with the stimulating agent, is between or between about 1 hour and 96 hours, 1 hour and 72 hours, 1 hour and 48 hours, 4 hours and 36 hours, 8 hours and 30 hours or 12 hours and 24 hours, such as at least or about at least 6 hours, 12 hours, 18 hours, 24 hours, 36 hours or 72 hours. In some embodiments, the further incubation is for a time between or about between 1 hour and 48 hours, 4 hours and 36 hours, 8 hours and 30 hours or 12 hours and 24 hours, inclusive.

4. Formulation

In some embodiments, one or more process steps (e.g. carried out in the centrifugal chamber and/or closed system) for manufacturing, generating or producing a cell therapy and/or engineered cells may include formulation of cells, such as formulation of genetically engineered cells resulting from the provided transduction processing steps prior to or after the culturing, e.g. cultivation and expansion, and/or one or more other processing steps as described. In some embodiments, the provided methods associated with formulation of cells include processing transduced cells, such as cells transduced and/or expanded using the processing steps described above, in a closed system.

In some embodiments, T cells, such as CD4+ and/or CD8+ T cells, generated by one or more of the processing steps are formulated. In some aspects, a plurality of compositions are separately manufactured, produced or generated, each containing a different population and/or sub-types of cells from the subject, such as for administration separately or independently, optionally within a certain period of time. For example, separate formulations of engineered cells containing different populations or sub-types of cells can include CD8⁺ and CD4⁺ T cells, respectively, and/or CD8+- and CD4+-enriched populations, respectively, e.g., CD4+ and/or CD8+ T cells each individually including cells genetically engineered to express the recombinant receptor. In some embodiments, at least one composition is formulated with comprises CD4+ T cells genetically engineered to express the recombinant receptor. In some embodiments, at least one composition is formulated with CD8+ T cells genetically engineered to express the recombinant receptor. In some embodiments, the administration of the dose comprises administration of a first composition comprising a dose of CD8+ T cells or a dose of CD4+ T cells and administration of a second composition comprising the other of the dose of CD4+ T cells and the CD8+ T cells. In some embodiments, a first composition comprising a dose of CD8+ T cells or a dose of CD4+ T cells is administered prior to the second composition comprising the other of the dose of CD4+ T cells and the CD8+ T cells. In some embodiments, the administration of the dose comprises administration of a composition comprising both of a dose of CD8+ T cells and a dose of CD4+ T cells.

In some embodiments, the cells are formulated in a pharmaceutically acceptable buffer, which may, in some aspects, include a pharmaceutically acceptable carrier or excipient. In some embodiments, the processing includes exchange of a medium into a medium or formulation buffer that is pharmaceutically acceptable or desired for administration to a subject. In some embodiments, the processing steps can involve washing the transduced and/or expanded cells to replace the cells in a pharmaceutically acceptable buffer that can include one or more optional pharmaceutically acceptable carriers or excipients. Exemplary of such pharmaceutical forms, including pharmaceutically acceptable carriers or excipients, can be any described below in conjunction with forms acceptable for administering the cells and compositions to a subject. The pharmaceutical composition in some embodiments contains the cells in amounts effective to treat or prevent the disease or condition, such as a therapeutically effective or prophylactically effective amount.

A “pharmaceutically acceptable carrier” refers to an ingredient in a pharmaceutical formulation, other than an active ingredient, which is nontoxic to a subject. A pharmaceutically acceptable carrier includes, but is not limited to, a buffer, excipient, stabilizer, or preservative.

In some aspects, the choice of carrier is determined in part by the particular cell and/or by the method of administration. Accordingly, there are a variety of suitable formulations. For example, the pharmaceutical composition can contain preservatives. Suitable preservatives may include, for example, methylparaben, propylparaben, sodium benzoate, and benzalkonium chloride. In some aspects, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

Buffering agents in some aspects are included in the compositions. Suitable buffering agents include, for example, citric acid, sodium citrate, phosphoric acid, potassium phosphate, and various other acids and salts. In some aspects, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition may also contain more than one active ingredient useful for the particular indication, disease, or condition being treated with the cells, preferably those with activities complementary to the cells, where the respective activities do not adversely affect one another. Such active ingredients are suitably present in combination in amounts that are effective for the purpose intended. Thus, in some embodiments, the pharmaceutical composition further includes other pharmaceutically active agents or drugs, such as chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

Compositions in some embodiments are provided as sterile liquid preparations, e.g., isotonic aqueous solutions, suspensions, emulsions, dispersions, or viscous compositions, which may in some aspects be buffered to a selected pH. Liquid compositions can comprise carriers, which can be a solvent or dispersing medium containing, for example, water, saline, phosphate buffered saline, polyol (for example, glycerol, propylene glycol, liquid polyethylene glycol) and suitable mixtures thereof. Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts may in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

In some embodiments, the formulation buffer contains a cryopreservative. In some embodiments, the cell are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the processing steps can involve washing the transduced and/or expanded cells to replace the cells in a cryopreservative solution.

In some embodiments, the formulation is carried out using one or more processing step including washing, diluting or concentrating the cells, such as the cultured or expanded cells. In some embodiments, the processing can include dilution or concentration of the cells to a desired concentration or number, such as unit dose form compositions including the number of cells for administration in a given dose or fraction thereof. In some embodiments, the processing steps can include a volume-reduction to thereby increase the concentration of cells as desired. In some embodiments, the processing steps can include a volume-addition to thereby decrease the concentration of cells as desired. In some embodiments, the processing includes adding a volume of a formulation buffer to transduced and/or expanded cells. In some embodiments, the volume of formulation buffer is from or from about 10 mL to 1000 mL, such as at least or about at least or about or 50 mL, 100 mL, 200 mL, 300 mL, 400 mL, 500 mL, 600 mL, 700 mL, 800 mL, 900 mL or 1000 mL.

In some embodiments, such processing steps for formulating a cell composition is carried out in a closed system. Exemplary of such processing steps can be performed using a centrifugal chamber in conjunction with one or more systems or kits associated with a cell processing system, such as a centrifugal chamber produced and sold by Biosafe SA, including those for use with the Sepax® or Sepax 2® cell processing systems. An exemplary system and process is described in International Publication Number WO2016/073602. In some embodiments, the method includes effecting expression from the internal cavity of the centrifugal chamber a formulated composition, which is the resulting composition of cells formulated in a formulation buffer, such as pharmaceutically acceptable buffer, in any of the above embodiments as described. In some embodiments, the expression of the formulated composition is to a container, such as a bag that is operably linked as part of a closed system with the centrifugal chamber. In some embodiments, the container, such as bag, is connected to a system at an output line or output position.

In some embodiments, the closed system, such as associated with a centrifugal chamber or cell processing system, includes a multi-port output kit containing a multi-way tubing manifold associated at each end of a tubing line with a port to which one or a plurality of containers can be connected for expression of the formulated composition. In some aspects, a desired number or plurality of output containers, e.g., bags, can be sterilely connected to one or more, generally two or more, such as at least 3, 4, 5, 6, 7, 8 or more of the ports of the multi-port output. For example, in some embodiments, one or more containers, e.g., bags can be attached to the ports, or to fewer than all of the ports. Thus, in some embodiments, the system can effect expression of the output composition into a plurality of output bags. In some aspects, cells can be expressed to the one or more of the plurality of output bags in an amount for dosage administration, such as for a single unit dosage administration or multiple dosage administration. For example, in some embodiments, the output bags may each contain the number of cells for administration in a given dose or fraction thereof. Thus, each bag, in some aspects, may contain a single unit dose for administration or may contain a fraction of a desired dose such that more than one of the plurality of output bags, such as two of the output bags, or 3 of the output bags, together constitute a dose for administration.

Thus, the containers, e.g., output bags, generally contain the cells to be administered, e.g., one or more unit doses thereof. The unit dose may be an amount or number of the cells to be administered to the subject or twice the number (or more) of the cells to be administered. It may be the lowest dose or lowest possible dose of the cells that would be administered to the subject.

In some embodiments, each of the containers, e.g., bags, individually comprises a unit dose of the cells. Thus in some embodiments, each of the containers comprises the same or approximately or substantially the same number of cells. In some embodiments, each unit dose contains at least or about at least 1×10⁶, 2×10⁶, 5×10⁶, 1×10⁷, 5×10⁷, or 1×10⁸ engineered cells, total cells, T cells, or PBMCs. In some embodiments, the volume of the formulated cell composition in each bag is 10 mL to 100 mL, such as at least or about at least 20 mL, 30 mL, 40 mL, 50 mL, 60 mL, 70 mL, 80 mL, 90 mL or 100 mL.

In some embodiments, such cells produced by the method, or a composition comprising such cells, are administered to a subject for treating a disease or condition.

### III. Recombinant Protein

In some embodiments, the methods for culturing, such as for expansion or cultivation of cells, is carried out on cells genetically engineered, e.g. transduced, with a recombinant protein. In some embodiments, the recombinant protein is or includes a recombinant receptor, e.g. an antigen receptor. The antigen receptor may include a functional non-TCR antigen receptors, including chimeric antigen receptors (CARs), and other antigen-binding receptors such as transgenic T cell receptors (TCRs). The receptors may also include other receptors, such as other chimeric receptors, such as receptors that bind to particular ligands and having transmembrane and/or intracellular signaling domains similar to those present in a CAR.

Exemplary antigen receptors, including CARs, and methods for engineering and introducing such receptors into cells, include those described, for example, in international patent application publication numbers WO200014257, WO2013126726, WO2012/129514, WO2014031687, WO2013/166321, WO2013/071154, WO2013/123061, U.S. patent application publication numbers US2002131960, US2013287748, US20130149337, U.S. Pat. Nos. 6,451,995, 7,446,190, 8,252,592, 8,339,645, 8,398,282, 7,446,179, 6,410,319, 7,070,995, 7,265,209, 7,354,762, 7,446,191, 8,324,353, and 8,479,118, and European patent application number EP2537416, and/or those described by Sadelain et al., Cancer Discov. 2013 April; 3(4): 388-398; Davila et al. (2013) PLoS ONE 8(4): e61338; Turtle et al., Curr. Opin. Immunol., 2012 October; 24(5): 633-39; Wu et al., Cancer, 2012 Mar. 18(2): 160-75. In some aspects, the antigen receptors include a CAR as described in U.S. Pat. No. 7,446,190, and those described in International Patent Application Publication No.: WO/2014055668 A1. Examples of the CARs include CARs as disclosed in any of the aforementioned publications, such as WO2014031687, U.S. Pat. Nos. 8,339,645, 7,446,179, US 2013/0149337, U.S. Pat. Nos. 7,446,190, 8,389,282, Kochenderfer et al., 2013, Nature Reviews Clinical Oncology, 10, 267-276 (2013); Wang et al. (2012) J. Immunother. 35(9): 689-701; and Brentjens et al., Sci Transl Med. 2013 5(177). See also WO2014031687, U.S. Pat. Nos. 8,339,645, 7,446,179, US 2013/0149337, U.S. Pat. Nos. 7,446,190, and 8,389,282.

In some embodiments, the nucleic acid(s) encoded the recombinant protein further encodes one or more marker, e.g., for purposes of confirming transduction or engineering of the cell to express the receptor and/or selection and/or targeting of cells expressing molecule(s) encoded by the polynucleotide. In some aspects, such a marker may be encoded by a different nucleic acid or polynucleotide, which also may be introduced during the genetic engineering process, typically via the same method, e.g., transduction by any of the methods provided herein, e.g., via the same vector or type of vector.

In some aspects, the marker, e.g., transduction marker, is a protein and/or is a cell surface molecule. Exemplary markers are truncated variants of a naturally-occurring, e.g., endogenous markers, such as naturally-occurring cell surface molecules. In some aspects, the variants have reduced immunogenicity, reduced trafficking function, and/or reduced signaling function compared to the natural or endogenous cell surface molecule. In some embodiments, the marker is a truncated version of a cell surface receptor, such as truncated EGFR (tEGFR). In some aspects, the marker includes all or part (e.g., truncated form) of CD34, an NGFR, or epidermal growth factor receptor (e.g., tEGFR). In some embodiments, the nucleic acid encoding the marker is operably linked to a polynucleotide encoding for a linker sequence, such as a cleavable linker sequence, e.g., T2A P2A, E2A and/or F2A. See, e.g., WO2014/031687.

In some embodiments, the marker is a molecule, e.g., cell surface protein, not naturally found on T cells or not naturally found on the surface of T cells, or a portion thereof.

In some embodiments, the molecule is a non-self molecule, e.g., non-self protein, i.e., one that is not recognized as “self” by the immune system of the host into which the cells will be adoptively transferred.

In some embodiments, the marker serves no therapeutic function and/or produces no effect other than to be used as a marker for genetic engineering, e.g., for selecting cells successfully engineered. In other embodiments, the marker may be a therapeutic molecule or molecule otherwise exerting some desired effect, such as a ligand for a cell to be encountered in vivo, such as a costimulatory or immune checkpoint molecule to enhance and/or dampen responses of the cells upon adoptive transfer and encounter with ligand.

A. Chimeric Antigen Receptors

In some embodiments, a CAR is generally a genetically engineered receptor with an extracellular ligand binding domain, such as an extracellular portion containing an antibody or fragment thereof, linked to one or more intracellular signaling components. In some embodiments, the chimeric antigen receptor includes a transmembrane domain and/or intracellular domain linking the extracellular domain and the intracellular signaling domain. Such molecules typically mimic or approximate a signal through a natural antigen receptor and/or signal through such a receptor in combination with a costimulatory receptor.

In some embodiments, CARs are constructed with a specificity for a particular marker, such as a marker expressed in a particular cell type to be targeted by adoptive therapy, e.g., a cancer marker and/or any of the antigens described. Thus, the CAR typically includes one or more antigen-binding fragment, domain, or portion of an antibody, or one or more antibody variable domains, and/or antibody molecules. In some embodiments, the CAR includes an antigen-binding portion or portions of an antibody molecule, such as a variable heavy chain (VH) or antigen-binding portion thereof, or a single-chain antibody fragment (scFv) derived from the variable heavy (VH) and variable light (VL) chains of a monoclonal antibody (mAb).

In some embodiments, the CAR contains an antibody or an antigen-binding fragment (e.g. scFv) that specifically recognizes an antigen, such as an intact antigen, expressed on the surface of a cell.

In some embodiments, the antigen (or a ligand) is a tumor antigen or cancer marker. In some embodiments, the antigen (or a ligand) is or includes orphan tyrosine kinase receptor ROR1, B cell maturation antigen (BCMA), carbonic anhydrase 9 (CAIX), tEGFR, Her2/neu (receptor tyrosine kinase erbB2), L1-CAM, CD19, CD20, CD22, mesothelin, CEA, and hepatitis B surface antigen, anti-folate receptor, CD23, CD24, CD30, CD33, CD38, CD44, EGFR, epithelial glycoprotein 2 (EPG-2), epithelial glycoprotein 40 (EPG-40), EPHa2, erb-B2, erb-B3, erb-B4, erbB dimers, EGFR vIII, folate binding protein (FBP), FCRL5, FCRH5, fetal acetylcholine receptor, GD2, GD3, HMW-MAA, IL-22R-alpha, IL-13R-alpha2, kinase insert domain receptor (kdr), kappa light chain, Lewis Y, L1-cell adhesion molecule, (L1-CAM), Melanoma-associated antigen (MAGE)-A1, MAGE-A3, MAGE-A6, Preferentially expressed antigen of melanoma (PRAME), survivin, TAG72, B7-H6, IL-13 receptor alpha 2 (IL-13Ra2), CA9, GD3, HMW-MAA, CD171, G250/CAIX, HLA-AI MAGE Al, HLA-A2 NY-ESO-1, PSCA, folate receptor-a, CD44v6, CD44v7/8, avb6 integrin, 8H9, NCAM, VEGF receptors, 5T4, Foetal AchR, NKG2D ligands, CD44v6, dual antigen, a cancer-testes antigen, mesothelin, murine CMV, mucin 1 (MUC1), MUC16, PSCA, NKG2D, NY-ESO-1, MART-1, gp100, oncofetal antigen, ROR1, TAG72, VEGF-R2, carcinoembryonic antigen (CEA), Her2/neu, estrogen receptor, progesterone receptor, ephrinB2, CD123, c-Met, GD-2, O-acetylated GD2 (OGD2), CE7, Wilms Tumor 1 (WT-1), a cyclin, cyclin A2, CCL-1, CD138, a pathogen-specific antigen and an antigen associated with a universal tag, and/or biotinylated molecules, and/or molecules expressed by HIV, HCV, HBV or other pathogens. Antigens targeted by the receptors in some embodiments include antigens associated with a B cell malignancy, such as any of a number of known B cell marker. In some embodiments, the antigen targeted by the receptor is CD20, CD19, CD22, ROR1, CD45, CD21, CD5, CD33, Igkappa, Iglambda, CD79a, CD79b or CD30.

In some embodiments, the antigen is a pathogen-specific antigen. In some embodiments, the antigen is a viral antigen (such as a viral antigen from HIV, HCV, HBV, etc.), bacterial antigens, and/or parasitic antigens.

In some embodiments, the CAR contains a TCR-like antibody, such as an antibody or an antigen-binding fragment (e.g. scFv) that specifically recognizes an intracellular antigen, such as a tumor-associated antigen, presented on the cell surface as a MHC-peptide complex. In some embodiments, an antibody or antigen-binding portion thereof that recognizes an MHC-peptide complex can be expressed on cells as part of a recombinant receptor, such as an antigen receptor. Among the antigen receptors are functional non-TCR antigen receptors, such as chimeric antigen receptors (CARs). Generally, a CAR containing an antibody or antigen-binding fragment that exhibits TCR-like specificity directed against peptide-MHC complexes also may be referred to as a TCR-like CAR.

In some embodiments, the extracellular portion of the CAR, such as an antibody portion thereof, further includes a spacer, such as a spacer region between the antigen-recognition component, e.g. scFv, and a transmembrane domain. The spacer may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. The spacer can be of a length that provides for increased responsiveness of the cell following antigen binding, as compared to in the absence of the spacer. In some examples, the spacer is at or about 12 amino acids in length or is no more than 12 amino acids in length. Exemplary spacers include those having at least about 10 to 229 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. In some embodiments, a spacer region has about 12 amino acids or less, about 119 amino acids or less, or about 229 amino acids or less. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or international patent application publication number WO2014/031687.

The extracellular ligand binding, such as antigen recognition domain, generally is linked to one or more intracellular signaling components, such as signaling components that mimic activation through an antigen receptor complex, such as a TCR complex, in the case of a CAR, and/or signal via another cell surface receptor. In some embodiments, a transmembrane domain links the extracellular ligand binding and intracellular signaling domains. In some embodiments, the CAR includes a transmembrane domain fused to the extracellular domain. In one embodiment, a transmembrane domain that naturally is associated with one of the domains in the receptor, e.g., CAR, is used. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 or CD154. The transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain. In some embodiments, the linkage is by linkers, spacers, and/or transmembrane domain(s).

In some embodiments, a short oligo- or polypeptide linker, for example, a linker of between 2 and 10 amino acids in length, such as one containing glycines and serines, e.g., glycine-serine doublet, is present and forms a linkage between the transmembrane domain and the cytoplasmic signaling domain of the CAR.

The recombinant receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 zeta chain. Thus, in some aspects, the antigen-binding portion is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor y, CD8, CD4, CD25, or CD16. For example, in some aspects, the CAR or other chimeric receptor includes a chimeric molecule between CD3-zeta (CD3-ζ) or Fc receptor y and CD8, CD4, CD25 or CD16.

In some embodiments, upon ligation of the CAR or other chimeric receptor, the cytoplasmic domain or intracellular signaling domain of the receptor activates at least one of the normal effector functions or responses of the immune cell, e.g., T cell engineered to express the CAR. For example, in some contexts, the CAR induces a function of a T cell such as cytolytic activity or T-helper activity, such as secretion of cytokines or other factors. In some embodiments, a truncated portion of an intracellular signaling domain of an antigen receptor component or costimulatory molecule is used in place of an intact immunostimulatory chain, for example, if it transduces the effector function signal. In some embodiments, the intracellular signaling domain or domains include the cytoplasmic sequences of the T cell receptor (TCR), and in some aspects also those of co-receptors that in the natural context act in concert with such receptors to initiate signal transduction following antigen receptor engagement, and/or any derivative or variant of such molecules, and/or any synthetic sequence that has the same functional capability.

In the context of a natural TCR, full activation generally requires not only signaling through the TCR, but also a costimulatory signal. Thus, in some embodiments, to promote full activation, a component for generating secondary or co-stimulatory signal is also included in the CAR. In other embodiments, the CAR does not include a component for generating a costimulatory signal. In some aspects, an additional CAR is expressed in the same cell and provides the component for generating the secondary or costimulatory signal.

T cell activation is in some aspects described as being mediated by at least two classes of cytoplasmic signaling sequences: those that initiate antigen-dependent primary activation through the TCR (primary cytoplasmic signaling sequences), and those that act in an antigen-independent manner to provide a secondary or co-stimulatory signal (secondary cytoplasmic signaling sequences). In some aspects, the CAR includes one or both of such signaling components.

In some aspects, the CAR includes a primary cytoplasmic signaling sequence that regulates primary activation of the TCR complex. Primary cytoplasmic signaling sequences that act in a stimulatory manner may contain signaling motifs which are known as immunoreceptor tyrosine-based activation motifs or ITAMs. Examples of ITAM containing primary cytoplasmic signaling sequences include those derived from TCR zeta, FcR gamma, FcR beta, CD3 gamma, CD3 delta, CD3 epsilon, CD8, CD22, CD79a, CD79b, and CD66d. In some embodiments, cytoplasmic signaling molecule(s) in the CAR contain(s) a cytoplasmic signaling domain, portion thereof, or sequence derived from CD3 zeta.

In some embodiments, the CAR includes a signaling domain and/or transmembrane portion of a costimulatory receptor, such as CD28, 4-1BB, OX40, CD27, DAP10, and ICOS. In some aspects, the same CAR includes both the activating and costimulatory components.

In some embodiments, the activating domain is included within one CAR, whereas the costimulatory component is provided by another CAR recognizing another antigen. In some embodiments, the CARs include activating or stimulatory CARs, and costimulatory CARs, both expressed on the same cell (see WO2014/055668). In some aspects, the CAR is the stimulatory or activating CAR; in other aspects, it is the costimulatory CAR. In some embodiments, the cells further include inhibitory CARs (iCARs, see Fedorov et al., Sci. Transl. Medicine, 5(215) (December, 2013), such as a CAR recognizing a different antigen, whereby an activating signal delivered through a CAR recognizing a first antigen is diminished or inhibited by binding of the inhibitory CAR to its ligand, e.g., to reduce off-target effects.

In some embodiments, the intracellular signaling domain of the CD8⁺ cytotoxic T cells is the same as the intracellular signaling domain of the CD4⁺ helper T cells. In some embodiments, the intracellular signaling domain of the CD8⁺ cytotoxic T cells is different than the intracellular signaling domain of the CD4⁺ helper T cells.

In certain embodiments, the intracellular signaling region comprises a CD28 transmembrane and signaling domain linked to a CD3 (e.g., CD3-zeta) intracellular domain. In some embodiments, the intracellular signaling region comprises a chimeric CD28 and CD137 (4-1BB, TNFRSF9) co-stimulatory domains, linked to a CD3 zeta intracellular domain.

In some embodiments, the CAR encompasses one or more, e.g., two or more, costimulatory domains and an activation domain, e.g., primary activation domain, in the cytoplasmic portion. Exemplary CARs include intracellular components of CD3-zeta, CD28, and 4-1BB.

In some cases, CARs are referred to as first, second, and/or third generation CARs. In some aspects, a first generation CAR is one that solely provides a CD3-chain induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In some embodiments, the chimeric antigen receptor includes an extracellular ligand-binding portion, such as an antigen-binding portion, such as an antibody or fragment thereof and in intracellular domain. In some embodiments, the antibody or fragment includes an scFv or a single-domain VH antibody and the intracellular domain contains an ITAM. In some aspects, the intracellular signaling domain includes a signaling domain of a zeta chain of a CD3-zeta (CD3ζ) chain. In some embodiments, the chimeric antigen receptor includes a transmembrane domain linking the extracellular domain and the intracellular signaling domain. In some aspects, the transmembrane domain contains a transmembrane portion of CD28. The extracellular domain and transmembrane can be linked directly or indirectly. In some embodiments, the extracellular domain and transmembrane are linked by a spacer, such as any described herein. In some embodiments, the chimeric antigen receptor contains an intracellular domain of a T cell costimulatory molecule, such as between the transmembrane domain and intracellular signaling domain. In some aspects, the T cell costimulatory molecule is CD28 or 4-1BB.

In some embodiments, the CAR contains an antibody, e.g., an antibody fragment, a transmembrane domain that is or contains a transmembrane portion of CD28 or a functional variant thereof, and an intracellular signaling domain containing a signaling portion of CD28 or functional variant thereof and a signaling portion of CD3 zeta or functional variant thereof. In some embodiments, the CAR contains an antibody, e.g., antibody fragment, a transmembrane domain that is or contains a transmembrane portion of CD28 or a functional variant thereof, and an intracellular signaling domain containing a signaling portion of a 4-1BB or functional variant thereof and a signaling portion of CD3 zeta or functional variant thereof. In some such embodiments, the receptor further includes a spacer containing a portion of an Ig molecule, such as a human Ig molecule, such as an Ig hinge, e.g. an IgG4 hinge, such as a hinge-only spacer.

In some embodiments, the transmembrane domain of the receptor, e.g., the CAR is a transmembrane domain of human CD28 or variant thereof, e.g., a 27-amino acid transmembrane domain of a human CD28 (Accession No.: P10747.1). In some embodiments, the intracellular domain comprises an intracellular costimulatory signaling domain of human CD28 or functional variant thereof, such as a 41 amino acid domain thereof and/or such a domain with an LL to GG substitution at positions 186-187 of a native CD28 protein. In some embodiments, the intracellular domain comprises an intracellular costimulatory signaling domain of 4-1BB or functional variant thereof, such as a 42-amino acid cytoplasmic domain of a human 4-1BB (Accession No. Q07011.1). In some embodiments, the intracellular signaling domain comprises a human CD3 zeta stimulatory signaling domain or functional variant thereof, such as an 112 AA cytoplasmic domain of isoform 3 of human CD3 (Accession No.: P20963.2) or a CD3 zeta signaling domain as described in U.S. Pat. No. 7,446,190. In some aspects, the spacer contains only a hinge region of an IgG, such as only a hinge of IgG4 or IgG1. In other embodiments, the spacer is an Ig hinge, e.g., and IgG4 hinge, linked to a CH2 and/or CH3 domains. In some embodiments, the spacer is an Ig hinge, e.g., an IgG4 hinge, linked to CH2 and CH3 domains. In some embodiments, the spacer is an Ig hinge, e.g., an IgG4 hinge, linked to a CH3 domain only. In some embodiments, the spacer is or comprises a glycine-serine rich sequence or other flexible linker such as known flexible linkers.

For example, in some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof; an intracellular signaling domain containing a signaling portion of CD28 or functional variant thereof; and a signaling portion of CD3 zeta signaling domain or functional variant thereof. In some embodiments, the CAR includes: an extracellular ligand-binding portion, such as an antigen-binding portion, such as an antibody or fragment thereof, including sdAbs and scFvs, that specifically binds an antigen, e.g. an antigen described herein; a spacer such as any of the Ig-hinge containing spacers; a transmembrane domain that is a portion of CD28 or a variant thereof; an intracellular signaling domain containing a signaling portion of 4-1BB or functional variant thereof; and a signaling portion of CD3 zeta signaling domain or functional variant thereof. In some embodiments, such CAR constructs further includes a T2A ribosomal skip element and/or a tEGFR sequence, e.g., downstream of the CAR.

B. T Cell Receptors (TCRs)

In some embodiments, the recombinant protein is or include a recombinant T cell receptor (TCR). In some embodiments, the recombinant TCR is specific for an antigen, generally an antigen present on a target cell, such as a tumor-specific antigen, an antigen expressed on a particular cell type associated with an autoimmune or inflammatory disease, or an antigen derived from a viral pathogen or a bacterial pathogen.

In some embodiments, the TCR is one that has been cloned from naturally occurring T cells. In some embodiments, a high-affinity T cell clone for a target antigen (e.g., a cancer antigen) is identified and isolated from a patient. In some embodiments, the TCR clone for a target antigen has been generated in transgenic mice engineered with human immune system genes (e.g., the human leukocyte antigen system, or HLA). See, e.g., tumor antigens (see, e.g., Parkhurst et al. (2009) Clin Cancer Res. 15:169-180 and Cohen et al. (2005) J Immunol. 175:5799-5808. In some embodiments, phage display is used to isolate TCRs against a target antigen (see, e.g., Varela-Rohena et al. (2008) Nat Med. 14:1390-1395 and Li (2005) Nat Biotechnol. 23:349-354.

In some embodiments, after the T-cell clone is obtained, the TCR alpha and beta chains are isolated and cloned into a gene expression vector. In some embodiments, the TCR alpha and beta genes are linked via a picornavirus 2A ribosomal skip peptide so that both chains are coexpressed. In some embodiments, the nucleic acid encoding a TCR further includes a marker to confirm transduction or engineering of the cell to express the receptor.

### IV. Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

As used herein, the singular forms “a,” “an,” and “the” include plural referents unless the context clearly dictates otherwise. For example, “a” or “an” means “at least one” or “one or more.” It is understood that aspects and variations described herein include “consisting” and/or “consisting essentially of” aspects and variations. It is also to be understood that the term “and/or” as used herein refers to and encompasses any and all possible combinations of one or more of the associated listed items. It is further to be understood that the terms “includes, “including,” “comprises,” and/or “comprising,” when used herein, specify the presence of stated features, integers, steps, operations, elements, components, and/or units but do not preclude the presence or addition of one or more other features, integers, steps, operations, elements, components, units, and/or groups thereof.

This application discloses several numerical ranges in the text and figures. The numerical ranges disclosed inherently support any range or value within the disclosed numerical ranges, including the endpoints, even though a precise range limitation is not stated verbatim in the specification because this disclosure can be practiced throughout the disclosed numerical ranges.

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.” Among the vectors are viral vectors, such as retroviral, e.g., gammaretroviral and lentiviral vectors.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

As used herein, a statement that a cell or population of cells is “positive” for a particular marker refers to the detectable presence on or in the cell of a particular marker, typically a surface marker. When referring to a surface marker, the term refers to the presence of surface expression as detected by flow cytometry, for example, by staining with an antibody that specifically binds to the marker and detecting said antibody, wherein the staining is detectable by flow cytometry at a level substantially above the staining detected carrying out the same procedure with an isotype-matched control under otherwise identical conditions and/or at a level substantially similar to that for cell known to be positive for the marker, and/or at a level substantially higher than that for a cell known to be negative for the marker.

As used herein, a statement that a cell or population of cells is “negative” for a particular marker refers to the absence of substantial detectable presence on or in the cell of a particular marker, typically a surface marker. When referring to a surface marker, the term refers to the absence of surface expression as detected by flow cytometry, for example, by staining with an antibody that specifically binds to the marker and detecting said antibody, wherein the staining is not detected by flow cytometry at a level substantially above the staining detected carrying out the same procedure with an isotype-matched control under otherwise identical conditions, and/or at a level substantially lower than that for cell known to be positive for the marker, and/or at a level substantially similar as compared to that for a cell known to be negative for the marker.

As used herein, a composition refers to any mixture of two or more products, substances, or compounds, including cells. It may be a solution, a suspension, liquid, powder, a paste, aqueous, non-aqueous or any combination thereof.

As used herein, a “subject” is a mammal, such as a human or other animal, and typically is human.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section heading used herein are for organizational purposes only and are not to be construed as limiting the subject matter described.

### V. Exemplary Embodiments

Among the embodiments provided herein are:

1. A bioreactor bag assembly comprising:

a bioreactor bag comprising:

a top surface comprising a plurality of ports, wherein the plurality of ports comprises

a feed port, a sampling port, and a perfusion port;

a bottom surface;

a perfusion filter fluidly connected to the perfusion port; and

a waste bag fluidly connected to the perfusion port of the bioreactor bag.

2. The bioreactor bag assembly of embodiment 1, wherein the top surface of the bioreactor bag has a first end and a second end opposite the first end, and the perfusion port is closer to the second end than the first end.

3. The bioreactor bag assembly of embodiments 1-2, wherein the feed port and the sampling port are closer to the first end than the second end.

4. The bioreactor bag assembly of embodiments 1-3, wherein the top surface of the bioreactor bag has a first side and a second side opposite the first side, and the feed port is closer to the first side than the second side.

5. The bioreactor bag assembly of embodiment 4, wherein the sampling port is closer to the second side than the first side.

6. The bioreactor bag assembly of embodiments 4-5, wherein the perfusion port is closer to the second side than the first side.

7. The bioreactor bag assembly of embodiments 1-6, wherein the perfusion filter is inside the bioreactor bag.

8. The bioreactor bag assembly of embodiments 1-7, further comprising a feed tubing arrangement fluidly connected to the feed port.

9. The bioreactor bag assembly of embodiment 8, wherein the feed tubing arrangement comprises polyvinyl chloride (PVC) tubing.

10. The bioreactor bag assembly of embodiments 8-9, wherein the feed tubing arrangement comprises a Y-connector such that the feed tubing arrangement has two inlets.

11. The bioreactor bag assembly of embodiments 1-10, further comprising a sampling tubing arrangement fluidly connected to the sampling port.

12. The bioreactor bag assembly of embodiment 11, wherein the sampling tubing arrangement comprises PVC tubing.

13. The bioreactor bag assembly of embodiment 1-12, wherein the waste bag is fluidly connected to the perfusion port via a waste tubing arrangement.

14. The bioreactor bag assembly of embodiment 13, wherein the waste tubing arrangement comprises PVC tubing.

15. The bioreactor bag assembly of embodiment 1-14, wherein the plurality of ports further comprises a gas inlet port and a gas outlet port.

16. The bioreactor bag assembly of embodiment 15, wherein the top surface of the bioreactor bag has a middle that is halfway between the first end and the second end, and the gas inlet port and the gas outlet port are closer to the middle than the first end or second end.

17. The bioreactor bag assembly of embodiment 15-16, wherein the plurality of ports consists of the feed port, the sampling port, the perfusion port, the gas inlet port, and the gas outlet port.

18. The bioreactor bag assembly of embodiment 15-17, further comprising a gas inlet tubing arrangement comprising an inlet filter fluidly connected to the gas inlet port and a gas outlet tubing arrangement comprising an exhaust filter fluidly connected to the gas outlet port.

19. A bioreactor system comprising:
a bioreactor rocker; and
a bioreactor bag supported on the bioreactor rocker, the bioreactor bag comprising:
   a top surface comprising a plurality of ports, wherein the plurality of ports comprises a feed port, a sampling port, and a perfusion port;
   a bottom surface;
   a perfusion filter fluidly connected to the perfusion port; and
a waste bag fluidly connected to the perfusion port of the bioreactor bag.

20. The bioreactor system of embodiment 19, further comprising a feed tubing arrangement fluidly connected to the feed port.

21. The bioreactor system of embodiment 20, wherein the feed tubing arrangement comprises a Y-connector such that the feed tubing arrangement has a first and a second inlet.

22. The bioreactor system of embodiment 21, wherein a cell media source is fluidly connected to each inlet of the feed tubing arrangement.

23. The bioreactor system of embodiment 22, wherein each inlet comprises PVC and the cell media source is welded to the PVC of the inlet.

24. The bioreactor system of embodiment 21, wherein a cell source is fluidly connected to the first inlet and a cell media source is fluidly connected to the second inlet.

25. The bioreactor system of embodiment 24, wherein each inlet comprises PVC and the cell source is welded to the PVC of the first inlet and the cell media source is welded to the PVC of the second inlet.

26. The bioreactor system of embodiments 19-25, wherein the perfusion filter is inside the bioreactor bag.

27. The bioreactor system of embodiments 19-26, wherein the top surface of the bioreactor bag has a first end and a second end opposite the first end, and the perfusion port is closer to the second end than the first end.

28. The bioreactor system of embodiment 27, wherein the feed port and the sampling port are closer to the first end than the second end.

29 The bioreactor system of embodiments 19-28, wherein the top surface of the bioreactor bag has a first side and a second side opposite the first side, and the feed port is closer to the first side than the second side.

30. The bioreactor system of embodiment 29, wherein the sampling port is closer to the second side than the first side.

31. The bioreactor system of embodiment 29-30, wherein the perfusion port is closer to the second side than the first side.

32. The bioreactor system of embodiments 19-31, wherein the perfusion filter is inside the bioreactor bag.

33. The bioreactor system of embodiments 19-32, further comprising a feed tubing arrangement fluidly connected to the feed port.

34. The bioreactor system of embodiment 33, wherein the feed tubing arrangement comprises polyvinyl chloride (PVC) tubing.

35. The bioreactor system of embodiments 33-34, wherein the feed tubing arrangement comprises a Y-connector such that the feed tubing arrangement has two inlets.

36. The bioreactor system of embodiments 19-35, further comprising a sampling tubing arrangement fluidly connected to the sampling port.

37. The bioreactor system of embodiment 36, wherein the sampling tubing arrangement comprises PVC tubing.

38. The bioreactor system of embodiments 19-37, wherein the waste bag is fluidly connected to the perfusion port via a waste tubing arrangement.

39. The bioreactor system of embodiment 38, wherein the waste tubing arrangement comprises PVC tubing.

40. The bioreactor system of embodiments 19-39, wherein the plurality of ports further comprises a gas inlet port and a gas outlet port.

41. The bioreactor system of embodiment 40, wherein the top surface of the bioreactor bag has a middle that is halfway between the first end and the second end, and the gas inlet port and the gas outlet port are closer to the middle than the first end or second end.

42. The bioreactor system of embodiments 40-41, wherein the plurality of ports consists of the feed port, the sampling port, the perfusion port, the gas inlet port, and the gas outlet port.

43. The bioreactor system of embodiments 40-42, further comprising a gas inlet tubing arrangement comprising an inlet filter fluidly connected to the gas inlet port and a gas outlet tubing arrangement comprising an exhaust filter fluidly connected to the gas outlet port.

44. A method of using a bioreactor system comprising:
providing a bioreactor bag of a bioreactor bag assembly, wherein the bioreactor bag assembly comprises:
   the bioreactor bag with a top surface comprising a plurality of ports, wherein the plurality of ports comprises a feed port, a sampling port, and a perfusion port; a bottom surface; and a perfusion filter fluidly connected to the perfusion port;
   a waste bag fluidly connected to the perfusion port of the bioreactor bag; and
supplying cell media to the bioreactor bag through the feed port;
supplying cells to the bioreactor bag through the feed port;
cultivating the cells in the bioreactor bag using agitation provided from a bioreactor rocker;
transferring waste filtrate through the perfusion port to the waste bag; and
harvesting the cultivated cells.

45. The method of embodiment 44, wherein the bioreactor bag assembly comprises a feed tubing arrangement fluidly connected to the feed port, wherein the feed tubing arrangement comprises a Y-connector such that the feed tubing arrangement has a first inlet and a second inlet.

46. The method of embodiment 45, wherein the cell media is added by welded a cell media source to the first inlet.

47. The method of embodiment 45, wherein the cells are added by welding a cell source to the first inlet.

48. The method of embodiment 45, wherein the cell media is added by welding a cell media source to the first inlet and the cells are added by welding a cell source to the second inlet.

49. The method of embodiments 44-48, wherein the plurality of ports further comprises a gas inlet port and a gas outlet port.

50. The method of embodiment 49, further comprising supplying a gas for cell cultivation to the bioreactor bag through the gas inlet port.

51. The method of embodiment 50, further comprising removing a portion of the gas from the bioreactor bag as exhaust through the gas outlet port.

52. The method of embodiment 45, wherein the cultivated cells are harvested by welding a harvest bag to the first or second inlet and reversing the flow direction of the feed tubing arrangement.

53. The method of embodiments 44-52, wherein the plurality of ports comprises a gas inlet port, and the method further comprises inflating the bioreactor bag with a gas through the gas inlet port.

54. The method of embodiments 44-53, further comprising retrieving a sample of the cultivated cells through the sampling port.

### VI. Examples

The following examples are included for illustrative purposes only and are not intended to limit the scope of the invention.

### Example 1: Exemplary Process for Generating Engineered T Cells for Autologous Cell Therapy

This example describes an exemplary process utilizing a bioreactor bag assembly of any of the provided embodiments, e.g., the exemplary bioreactor bag assemblies illustrated by FIGS. 1-7, to prepare engineered T cells for autologous cell therapy according to certain embodiments provided herein.

Compositions containing CD4+ and CD8+ cells are isolated from human leukapheresis samples by immunoaffinity-based enrichment and cryofrozen. The CD4+ and CD8+ cells are subsequently thawed and transferred to a closed system under sterile conditions. The cells are cultured under stimulating conditions and then transduced with a viral vector, such as a retroviral vector or a lentiviral vector, encoding a recombinant receptor. The recombinant receptor may be a chimeric antigen receptor (CAR), such as an anti-CD19 CAR. After transduction, the cells are transferred into the bioreactor bag assembly connected to the closed system under sterile conditions for subsequent expansion.

The bioreactor bag assembly is connected to a bioreactor (e.g., a Xuri W25) that regulates cell culture conditions under a closed system. An expansion media containing one or more cytokines is added the cells. The bioreactor is capable of culturing the cells under static conditions or with perfusion, whereby fresh media gradually replaces used media at a constant rate. At least a portion of the cell culture expansion is performed with perfusion, such as with a rate of 290 ml/day, 580 ml/day, or 1160 ml/day. The bioreactor regulates the cell culture conditions by maintaining temperature at or near 37° C. and CO₂ levels at or near 5% with a steady air flow at or near 0.1 L/min. At least a portion of the cell culture expansion is performed with a rocking motion, such as at an angle of between 5° and 10°, such as 6°, at a constant rocking speed, such as a speed of between 5 and 15 RPM, such as 6 RMP or 10 RPM. The cells are expanded until they reach a threshold amount or cell density. When the threshold is achieved, the connections between the bioreactor bag and the bioreactor are sealed, and the bag is transferred for subsequent cell formulation, e.g., cryofreezing.

### Example 2: Exemplary Process for Generating Engineered CD4+ and CD8+ T Cell Compositions for Autologous Cell Therapy

This example describes an exemplary process utilizing a bioreactor bag assembly of any of the provided embodiments, e.g., the exemplary bioreactor bag assemblies illustrated by FIGS. 1-7, to prepare separate compositions of engineered CD4+ and CD8+ T cells for autologous cell therapy according to certain embodiments provided herein.

Separate compositions of CD4+ and CD8+ cells are isolated from human leukapheresis samples by immunoaffinity-based enrichment and cryofrozen. The CD4+ and CD8+ cells of the compositions are subsequently thawed and separately transferred to a closed system under sterile conditions. The CD4+ and CD8+ cells are cultured under stimulating conditions and then transduced with a viral vector, such as a retroviral vector or a lentiviral vector, encoding a recombinant receptor. The recombinant receptor may be a chimeric antigen receptor (CAR), such as an anti-CD19 CAR. After transduction, the CD4+ and CD8+ cells are separately transferred into bioreactor bag assemblies connected to the closed system under sterile conditions for subsequent expansion.

The bioreactor bag assemblies are connected to a bioreactor (e.g., a Xuri W25) that regulates cell culture conditions under a closed system. Expansion media containing one or more cytokines are added to the CD4+ and CD8+ cells. The expansion media added to the CD4+ cells and the CD8+ cells may be the same or different. At least a portion of the cell culture expansion is performed with perfusion, such as with a rate of 290 ml/day, 580 ml/day, or 1160 ml/day. The bioreactor regulates the cell culture conditions by maintaining temperature at or near 37° C. and CO₂ levels at or near 5% with a steady air flow at or near 0.1 L/min. At least a portion of the cell culture expansion is performed with a rocking motion, such as at an angle of between 5° and 10°, such as 6°, at a constant rocking speed, such as a speed of between 5 and 15 RPM, such as 6 RMP or 10 RPM. The CD4+ and CD8+ cells are each separately expanded until they reach a threshold amount or cell density. When the threshold is achieved, the connections between the bioreactor bag and the bioreactor are sealed, and the bag is transferred for subsequent cell formulation, e.g., cryofreezing.

The above description is presented to enable a person skilled in the art to make and use the disclosure, and is provided in the context of a particular application and its requirements. Various modifications to the preferred embodiments will be readily apparent to those skilled in the art, and the generic principles defined herein may be applied to other embodiments and applications without departing from the spirit and scope of the disclosure. Thus, this disclosure is not intended to be limited to the embodiments shown, but is to be accorded the widest scope consistent with the principles and features disclosed herein.

1. A bioreactor bag assembly comprising:
a bioreactor bag comprising:
a top surface comprising a plurality of ports, wherein the plurality of ports comprises a feed port, a sampling port, and a perfusion port;
a bottom surface;
a perfusion filter fluidly connected to the perfusion port; and
a waste bag fluidly connected to the perfusion port of the bioreactor bag.

2. The bioreactor bag assembly of claim 1, wherein the top surface of the bioreactor bag has a first end and a second end opposite the first end, and the perfusion port is closer to the second end than the first end.

3. The bioreactor bag assembly of claims 1-2, wherein the feed port and the sampling port are closer to the first end than the second end.

4. The bioreactor bag assembly of claims 1-3, wherein the top surface of the bioreactor bag has a first side and a second side opposite the first side, and the feed port is closer to the first side than the second side.

5. The bioreactor bag assembly of claim 4, wherein the sampling port is closer to the second side than the first side.

6. The bioreactor bag assembly of claims 4-5, wherein the perfusion port is closer to the second side than the first side.

7. The bioreactor bag assembly of claims 1-6, wherein the perfusion filter is inside the bioreactor bag.

8. The bioreactor bag assembly of claims 1-7, further comprising a feed tubing arrangement fluidly connected to the feed port.

9. The bioreactor bag assembly of claim 8, wherein the feed tubing arrangement comprises polyvinyl chloride (PVC) tubing.

10. The bioreactor bag assembly of claims 8-9, wherein the feed tubing arrangement comprises a Y-connector such that the feed tubing arrangement has two inlets.

11. The bioreactor bag assembly of claims 1-10, further comprising a sampling tubing arrangement fluidly connected to the sampling port.

12. The bioreactor bag assembly of claim 11, wherein the sampling tubing arrangement comprises PVC tubing.

13. The bioreactor bag assembly of claim 1-12, wherein the waste bag is fluidly connected to the perfusion port via a waste tubing arrangement.

14. The bioreactor bag assembly of claim 13, wherein the waste tubing arrangement comprises PVC tubing.

15. The bioreactor bag assembly of claim 1-14, wherein the plurality of ports further comprises a gas inlet port and a gas outlet port.

16. The bioreactor bag assembly of claim 15, wherein the top surface of the bioreactor bag has a middle that is halfway between the first end and the second end, and the gas inlet port and the gas outlet port are closer to the middle than the first end or second end.

17. The bioreactor bag assembly of claim 15-16, wherein the plurality of ports consists of the feed port, the sampling port, the perfusion port, the gas inlet port, and the gas outlet port.

18. The bioreactor bag assembly of claim 15-17, further comprising a gas inlet tubing arrangement comprising an inlet filter fluidly connected to the gas inlet port and a gas outlet tubing arrangement comprising an exhaust filter fluidly connected to the gas outlet port.

19. A bioreactor system comprising:
a bioreactor rocker; and
a bioreactor bag supported on the bioreactor rocker, the bioreactor bag comprising:
a top surface comprising a plurality of ports, wherein the plurality of ports comprises a feed port, a sampling port, and a perfusion port;
a bottom surface;
a perfusion filter fluidly connected to the perfusion port; and
a waste bag fluidly connected to the perfusion port of the bioreactor bag.

20. The bioreactor system of claim 19, further comprising a feed tubing arrangement fluidly connected to the feed port.

21. The bioreactor system of claim 20, wherein the feed tubing arrangement comprises a Y-connector such that the feed tubing arrangement has a first and a second inlet.

22. The bioreactor system of claim 21, wherein a cell media source is fluidly connected to each inlet of the feed tubing arrangement.

23. The bioreactor system of claim 22, wherein each inlet comprises PVC and the cell media source is welded to the PVC of the inlet.

24. The bioreactor system of claim 21, wherein a cell source is fluidly connected to the first inlet and a cell media source is fluidly connected to the second inlet.

25. The bioreactor system of claim 24, wherein each inlet comprises PVC and the cell source is welded to the PVC of the first inlet and the cell media source is welded to the PVC of the second inlet.

26. The bioreactor system of claims 19-25, wherein the perfusion filter is inside the bioreactor bag.

27. The bioreactor system of claims 19-26, wherein the top surface of the bioreactor bag has a first end and a second end opposite the first end, and the perfusion port is closer to the second end than the first end.

28. The bioreactor system of claim 27, wherein the feed port and the sampling port are closer to the first end than the second end.

29. The bioreactor system of claims 19-28, wherein the top surface of the bioreactor bag has a first side and a second side opposite the first side, and the feed port is closer to the first side than the second side.

30. The bioreactor system of claim 29, wherein the sampling port is closer to the second side than the first side.

31. The bioreactor system of claim 29-30, wherein the perfusion port is closer to the second side than the first side.

32. The bioreactor system of claims 19-31, wherein the perfusion filter is inside the bioreactor bag.

33. The bioreactor system of claims 19-32, further comprising a feed tubing arrangement fluidly connected to the feed port.

34. The bioreactor system of claim 33, wherein the feed tubing arrangement comprises polyvinyl chloride (PVC) tubing.

35. The bioreactor system of claims 33-34, wherein the feed tubing arrangement comprises a Y-connector such that the feed tubing arrangement has two inlets.

36. The bioreactor system of claims 19-35, further comprising a sampling tubing arrangement fluidly connected to the sampling port.

37. The bioreactor system of claim 36, wherein the sampling tubing arrangement comprises PVC tubing.

38. The bioreactor system of claims 19-37, wherein the waste bag is fluidly connected to the perfusion port via a waste tubing arrangement.

39. The bioreactor system of claim 38, wherein the waste tubing arrangement comprises PVC tubing.

40. The bioreactor system of claims 19-39, wherein the plurality of ports further comprises a gas inlet port and a gas outlet port.

41. The bioreactor system of claim 40, wherein the top surface of the bioreactor bag has a middle that is halfway between the first end and the second end, and the gas inlet port and the gas outlet port are closer to the middle than the first end or second end.

42. The bioreactor system of claims 40-41, wherein the plurality of ports consists of the feed port, the sampling port, the perfusion port, the gas inlet port, and the gas outlet port.

43. The bioreactor system of claims 40-42, further comprising a gas inlet tubing arrangement comprising an inlet filter fluidly connected to the gas inlet port and a gas outlet tubing arrangement comprising an exhaust filter fluidly connected to the gas outlet port.

44. A method of using a bioreactor system comprising:
providing a bioreactor bag of a bioreactor bag assembly, wherein the bioreactor bag assembly comprises:
the bioreactor bag with a top surface comprising a plurality of ports, wherein the plurality of ports comprises a feed port, a sampling port, and a perfusion port; a bottom surface; and a perfusion filter fluidly connected to the perfusion port;
a waste bag fluidly connected to the perfusion port of the bioreactor bag; and
supplying cell media to the bioreactor bag through the feed port;
supplying cells to the bioreactor bag through the feed port;
cultivating the cells in the bioreactor bag using agitation provided from a bioreactor rocker;
transferring waste filtrate through the perfusion port to the waste bag; and
harvesting the cultivated cells.

45. The method of claim 44, wherein the bioreactor bag assembly comprises a feed tubing arrangement fluidly connected to the feed port, wherein the feed tubing arrangement comprises a Y-connector such that the feed tubing arrangement has a first inlet and a second inlet.

46. The method of claim 45, wherein the cell media is added by welded a cell media source to the first inlet.

47. The method of claim 45, wherein the cells are added by welding a cell source to the first inlet.

48. The method of claim 45, wherein the cell media is added by welding a cell media source to the first inlet and the cells are added by welding a cell source to the second inlet.

49. The method of claims 44-48, wherein the plurality of ports further comprises a gas inlet port and a gas outlet port.

50. The method of claim 49, further comprising supplying a gas for cell cultivation to the bioreactor bag through the gas inlet port.

51. The method of claim 50, further comprising removing a portion of the gas from the bioreactor bag as exhaust through the gas outlet port.

52. The method of claim 45, wherein the cultivated cells are harvested by welding a harvest bag to the first or second inlet and reversing the flow direction of the feed tubing arrangement.

53. The method of claims 44-52, wherein the plurality of ports comprises a gas inlet port, and the method further comprises inflating the bioreactor bag with a gas through the gas inlet port.

54. The method of claims 44-53, further comprising retrieving a sample of the cultivated cells through the sampling port.
